(19) United States
(12) Patent Application Publication
(10) Pub. No.: US 2017/0204092 A1
(43) Pub. Date: Jul. 20, 2017

(54) 4,5-DIHYDROISOXAZOLE DERIVATIVES AS NAMPT INHIBITORS

(71) Applicant: AURIGENE DISCOVERY TECHNOLOGIES LIMITED, Banagalore (IN)

(72) Inventors: Dinesh CHIKKANNA, Bangalore (IN); Vinayak KHAIRNAR, Nashik (IN)

(21) Appl. No.: 15/324,640

(22) PCT Filed: Jul. 22, 2015
(86) PCT No.: PCT/IB2015/055546

§ 371 Date: Jan. 6, 2017

(30) Foreign Application Priority Data
Jul. 23, 2014 (IN) 3604/CHE/2014

(51) Int. Cl. C07D 471/04; A61K 31/501; C07D 413/14; A61K 31/444; A61K 45/06

(52) U.S. Cl. CPC C07D 471/04; A61K 31/444; A61K 45/06; C07D 413/14; A61K 31/501

(57) ABSTRACT

The present invention provides 4,5-dihydroisoxazole derivatives of formula (I), which may be therapeutically useful, more particularly as NAMPT inhibitors. wherein, ring A, L₁, L₂, X₁, X₂, X₃, Z, R₁, R₂, R₃, R₄, R₅, m, n, p and q have the meanings given in the specification and pharmaceutically acceptable salts thereof that are useful in the treatment and prevention of diseases or disorder, caused by an elevated level of nicotinamide phosphoribosyltransferase (NAMPT) in a mammal. The present invention also provides preparation of the compounds and pharmaceutical formulations comprising at least one of the substituted 4,5-di hydroisoxazole derivatives of formula (I) or a pharmaceutically acceptable salt thereof or a stereoisomer thereof.

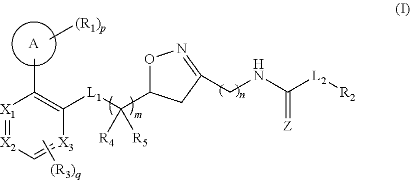

This application claims the benefit of Indian complete application 3604/CHE/2014 filed on 23^{rd} Jul. 2014 which hereby incorporated by reference.

### FIELD OF THE INVENTION

This invention relates to compounds useful for treatment of cancer and inflammatory diseases associated with nicotinamide phosphoribosyl transferase (NAMPT), and more particularly compounds that modulate the function of NAMPT. The invention also provides pharmaceutically acceptable compositions comprising compounds of the present invention and methods of using said compositions in the treatment of disease(s) and/or disorder(s) associated with the modulation of NAMPT.

### BACKGROUND OF THE INVENTION

NAD⁺ (nicotinamide adenine dinucleotide) is a coenzyme that plays a critical role in many physiologically essential processes (Ziegler, M. Eur. J. Biochem. 267, 1550-1564, 2000). NAD⁺ is necessary for several processes in signaling pathways including poly ADP-ribosylation in DNA repair, mono-ADP-ribosylation in both the immune response and G-protein-coupled signaling, deacylation mediated by sirtuins (Garten, A. et al Trends in Endocrinology and Metabolism, 20, 130-138, 2008).

NAMPT (also known as pre-B-cell-colony-enhancing factor (PBEF) and visfatin) is an enzyme that catalyzes the phosphoribosylation of nicotinamide and is the rate-limiting enzyme in one of two pathways that salvage NAD⁺.

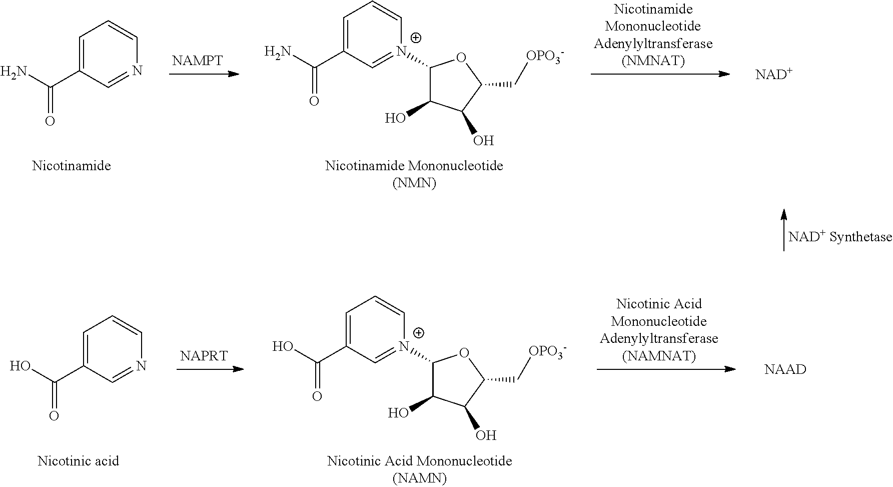

Increasing evidence suggests that NAMPT inhibitors have potential as anticancer agents. Cancer cells have a higher basal turnover of NAD⁺ and also display higher energy requirements compared with normal cells. Additionally, increased NAMPT expression has been reported in colorectal cancer (Van Beijnum, J. R. et al. Int. J. Cancer 101, 118-127, 2002) and NAMPT is involved in angiogenesis (Kim, S. R. et al. Biochem. Biophys. Res. Commun. 357, 150-156, 2007). Small-molecule inhibitors of NAMPT have been shown to cause depletion of intracellular NAD⁺ levels and ultimately induce tumor cell death (Hansen, C M et al. Anticancer Res. 20, 4211-4220, 2000) as well as inhibit tumor growth in xenograft models (Olesen, U. H. et al. Mol Cancer Ther. 9, 1609-1617, 2010).

NAMPT is prominently overexpressed in human prostate cancer cells along with SIRT1. Elevation of NAMPT expression occurs early for the prostate neoplasia. Inhibition of NAMPT significantly suppresses cell growth in culture, soft agar colony formation, cell invasion and growth of xenografted prostate cancer cells in mice (Wang, B. Et al. Oncogene, 30(8), 907-921, 2011). Further, NAMPT is a potent therapeutic target in the treatment of EGFR-gene-mutated NSCLC as well (Okumura, Shunsuke et al. Journal of Thoracic Oncology, 7(1), 49-56, 2012). It has also been revealed that NAMPT plays a novel role in the regulation of de novo lipogenesis through the modulation of sirtuin activity in prostate cancer (PCa) cells (Bowlby, Sarah C. Et al. PLoS One, 7(6), e40195, 2012).

NAMPT inhibitors also have potential as therapeutic agents in inflammatory and metabolic disorders (Galli, M. et al Cancer Res. 70, 8-11, 2010). For example, NAMPT is the predominant enzyme in T and B lymphocytes. Selective inhibition of NAMPT leads to NAD⁺ depletion in lymphocytes blocking the expansion that accompanies autoimmune disease progression whereas cell types expressing the other NAD⁺ generating pathways might be spared. A small molecule NAMPT inhibitor (FK866) has been shown to selectively block proliferation and induce apoptosis of activated T cells and was efficacious in animal models of arthritis (collagen-induced arthritis) (Busso, N. et al. PloS One 3(5), e2267, 2008). FK866 ameliorated the manifestations of experimental autoimmune encephalomyelitis (EAE), a model of T-cell mediated autoimmune disorders (Bruzzone, S et al. PloS One 4, e7897, 2009). As determined by MTT assay and flow cytometry, FK866 also increased the chemosensitivity of gastric cancer cells to fluorouracil by greater inhibition of cell proliferation and the induction of apoptosis (Bi, Tie-Qiang et al. Oncology Reports, 26(5), 1251-1257, 2011). NAMPT activity increases NF-kB transcriptional activity in human vascular endothelial cell, resulting in MMP-2 and MMP-9 activation, suggesting a role for NAMPT inhibitors in the prevention of inflammatory mediated complications of obesity and type 2 diabetes (Adya, R. et. al. Diabetes Care, 31, 758-760, 2008). The first NAMPT inhibitors (FK866 and CHS828) have already entered clinical trials, and a surge in interest in the synthesis of novel molecules has occurred (Journal of Medicinal Chemistry, 56(16), 6279-6296, 2013).

Inhibitors of NAMPT have been disclosed in WO2013082150, US 20120329786, WO2012177782, WO 2012154194, WO 2012150952, WO1997048696, WO 2013067710, US20120122842, US20120122924, WO2012031197, WO2012031196, WO2012031199, WO2012154194, WO2012150952, WO2011109441, WO2011121055, WO2009086835, WO2015100322 and in many other applications. Apparently, all of them are still in the early preclinical stage. There appears to be unmet need for newer drugs that can treat diseases and/or disorders associated with an elevated level of NAMPT. It is therefore, an object of this invention to provide compounds useful in the treatment and/or prevention or amelioration of such diseases and/or disorders associated with the modulation of NAMPT.

### SUMMARY OF THE INVENTION

Provided herein are 4,5-diydroisoxazole derivatives and pharmaceutical compositions thereof, which are useful as NAMPT inhibitors.

In one aspect of the present invention, it comprises compounds of formula (I):

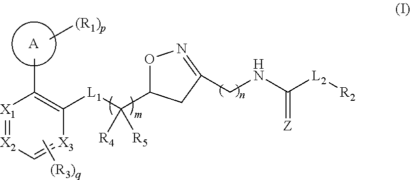

or a pharmaceutically acceptable salt or a stereoisomer thereof;
wherein,

ring A is carbocyclyl or heterocyclyl;

X₁, X₂ and X₃ independently are C or N; provided all X₁, X₂ and X₃ are not C at any instance;

L₁ is —O—, —S—, —SO—, —SO₂—, —NH—, —CO—, —NHCO—, —CONH—, —NHSO₂— or —SO₂NH—;

L₂ is a direct bond, —CH═CH— or —NR_{b}(CH₂)ᵣ—;

Z is O, S or NCN;

R₁ at each occurrence is independently alkyl, nitro, halo, haloalkyl, hydroxyalkyl, —ORₐ, —(CH₂)ₙNR_{b}R_{c}, —(SO₂)-alkyl, —(SO₂)—NR_{b}R_{c}, —NH(CO)alkyl or —(CO)NHR_{b};

R₂ is optionally substituted carbocyclyl or optionally substituted heterocyclyl wherein the optional substituent is R₆;

R₃ at each occurrence is hydrogen, halo, amino, nitro, cyano, alkyl, hydroxy, alkoxy, haloalkyl or haloalkoxy;

R₄ and R₅ independently are hydrogen or alkyl;

alternatively, R₄ and R₅ together with the carbon atom to which they are attached form an optionally substituted cycloalkyl ring; wherein the optional substituent is alkyl or halo;

R₆ at each occurrence is independently one or more alkyl, amino, halo, nitro, cyano, haloalkyl, hydroxyl, alkoxy, —NHSO₂-alkyl, cycloalkyl, optionally substituted aryl or optionally substituted heteroaryl wherein the optional substituent at each occurrence is halo, alkyl, hydroxyl, alkoxy or haloalkyl;

Rₐ is hydrogen, alkyl or arylalkyl;

R_{b} and R_{c} independently are hydrogen or alkyl;

m, n and p independently are 0, 1, 2 or 3;

q is 1, 2 or 3; and

r is 0, 1 or 2.

In yet another aspect, the present invention provides a pharmaceutical composition comprising the compound of formula (I) or a pharmaceutically acceptable salt or a stereoisomer thereof, and at least one pharmaceutically acceptable excipient (such as a pharmaceutically acceptable carrier or diluent).

In yet another aspect, the present invention relates to preparation of the compounds of formula (I).

In yet further aspect, the present invention provides use of a compound of formula (I) or a pharmaceutically acceptable salt or a stereoisomer thereof for the treatment and prevention of diseases and/or disorders caused by an elevated level of nicotinamide phosphoribosyltransferase (NAMPT).

More particularly, the invention relates to the use of compound of formula (I) or a pharmaceutically acceptable salt or a stereoisomer thereof including mixtures thereof in all ratios as a medicament, by inhibiting NAMPT.

The compounds of formula (I) of the present invention possess the therapeutic role of inhibiting NAMPT, which are useful in the treatment of diseases and/or disorders including, but not limited to the group consisting of cancer, pancreatic cancer, ovarian cancer, lung cancer, prostate cancer, skin cancer, breast cancer, uterine cancer, renal cancer, head and neck cancer, brain cancer, colon cancer, cervical cancer, bladder cancer, leukemia, lymphoma, Hodgkin's disease, viral infections including adult respiratory distress syndrome, ataxia telengiectasia, Human Immunodeficiency Virus, hepatitis virus, herpes virus, herpes simplex, inflammatory disorders, irritable bowel syndrome, inflammatory bowel disease, rheumatoid arthritis, asthma, chronic obstructive pulmonary disease, osteoarthritis, osteoporosis, fibrotic diseases, dermatosis, atopic dermatitis, psoriasis, ultra-violet induced skin damage, systemic lupus erythematosis, multiple sclerosis, psoriatic arthritis, ankylosing spondylitis, graft-versus-host disease, Alzheimer's disease, cerebrovascular accident, atherosclerosis, restenosis, diabetes, glomerulonephiritis, metabolic syndrome, non-small cell lung cancer, small cell lung cancer, multiple myeloma, leukemias, lymphomas, cancers of the brain and central nervous system, squamous cell cancers, kidney cancer, uretral and bladder cancers, cancers of head and neck.

### DETAILED DESCRIPTION OF THE INVENTION

Unless defined otherwise, all technical and scientific terms used herein have the same meaning as is commonly understood by one of skilled in the art to which the subject matter herein belongs. As used in the specification and the appended claims, unless specified to the contrary, the following terms have the meaning indicated in order to facilitate the understanding of the present invention.

As used herein the term “alkyl” refers to a hydrocarbon chain radical that includes solely carbon and hydrogen atoms in the backbone, containing no unsaturation, and which is attached to the rest of the molecule by a single bond. The alkane radical may be straight or branched. For example, the term “C₁-C₄ alkyl” refers to a monovalent, straight, or branched aliphatic group containing 1 to 4 carbon atoms (e.g., methyl, ethyl, n-propyl, i-propyl, n-butyl, i-butyl, s-butyl, t-butyl and the like).

As used herein, the term “alkoxy” refers to the radical —O-alkyl, wherein the alkyl is as defined above. Representative examples of alkoxy include, but are not limited to, methoxy, ethoxy, propoxy, 2-propoxy, n-butoxy, tert-butoxy, pentyloxy, hexyloxy and heptyloxy. The alkyl portion of the alkoxy may be optionally substituted.

As used herein the term “cycloalkyl” refers to C₃-C₁₀ saturated hydrocarbon ring. A cycloalkyl may be a single ring, which typically contains from 3 to 7 carbon ring atoms.

Examples of single-ring cycloalkyls include cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused, and spirocyclic cycloalkyls.

As used herein, the term “aryl” alone or in combination with other term(s) means a carbocyclic aromatic system containing one or more rings wherein such rings may be fused.

The term “fused” means that the second ring is attached or formed by having two adjacent atoms in common with the first ring. Unless otherwise specified, an aryl group typically has from 6 to about 14 carbon atoms but the invention is not limited in that respect. C₆-C₁₄ aryl refers to an aryl group having six to twelve carbon atoms. Examples of aryl groups include but are not limited to phenyl, naphthyl, indanyl, and the like. Unless otherwise specified, all aryl groups described herein may be optionally substituted.

As used herein, the term “carbocyclyl” alone or in combination with other term(s) refers to a saturated, partially saturated or unsaturated carbocyclic system. Carbocyclyl includes the definitions of both “cycloalkyl” and “aryl” groups as well, which are as defined above. Carbocyclyl ring can be monocyclic; or one or more rings can be fused to form bicyclic or polycyclic carbocyclyl ring such as aryl-fused-cycloalkyl or cycloalkyl-fused-aryl and the like. Examples of “carbocyclyl” includes, but are not limited to cyclopropyl, cyclobutyl, cyclopentyl, cyclohexyl, phenyl, naphthyl, cyclohexenyl, cyclopentenyl, 2,3-dihydro-1H-indene or 1,2,3,4-tetrahydronaphthalene and the like.

As used herein, the term “arylalkyl” refers to an alkyl group, as defined above, wherein one or more of the alkyl group's hydrogen atom has been replaced with an aryl group as defined above. Examples of arylalkyl group include, but are not limited to benzyl, benzhydryl, 1-phenylethyl, 2-phenylethyl, 3-phenylpropyl and the like. An arylalkyl group can be unsubstituted or substituted with one or more suitable groups.

“Cyano” refers to —CN group.

“Nitro” refers to —NO₂ group.

As used herein, the term “halo” or “halogen” alone or in combination with other term(s) means fluorine, chlorine, bromine or iodine.

As used herein, the term “amino” means —NH₂ group. This amino group can be optionally substituted with one or more alkyl groups, wherein alkyl groups are as defined above. Representative examples of an alkylamino group include, but not limited to —NHCH₃, —NHCH₂CH₃, —NHCH₂—CH(CH₃)₂, —N(CH₃)₂ and the like.

As used herein, the term “haloalkyl” means alkyl substituted with one or more halogen atoms, wherein the alkyl group is as defined above. The term “halo” is used herein interchangeably with the term “halogen” meaning F, Cl, Br or I. Examples of “haloalkyl” include but are not limited to fluoromethyl, difluoromethyl, trifluoromethyl, 2,2,2-trifluoroethyl and the like.

As used herein, the term “haloalkoxy” refers to —O-haloalkyl, wherein the haloalkyl is as defined above. Representative examples of haloalkoxy include, but are not limited to, trifluoromethoxy, chloromethoxy, 2-fluoroethoxy and the like.

“hydroxy” or “hydroxyl” refers to —OH group.

As used herein the term “hydroxyalkyl” means alkyl substituted with one or more hydroxy groups, where alkyl group is as defined above. Examples of “hydroxyalkyl” include but are not limited to hydroxymethyl, hydroxyethyl, hydroxypropyl, and the like.

The term “heteroaryl” unless otherwise specified refers to substituted or unsubstituted 5 to 14 membered aromatic ring radical with one or more heteroatom(s) independently selected from N, O or S (i.e. 5 to 14 membered heteroaryl). The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to oxazolyl, isoxazolyl, imidazolyl, furyl, indolyl, isoindolyl, pyrrolyl, triazolyl, triazinyl, tetrazoyl, thienyl, oxadiazolyl, thiazolyl, isothiazolyl, pyridyl, pyrimidinyl, pyrazinyl, pyridazinyl, pyrazolyl, benzofuranyl, benzothiazolyl, benzoxazolyl, benzimidazolyl, benzothienyl, benzopyranyl, carbazolyl, imidazo[1,2-a]pyridinyl and the like. Unless set forth or recited to the contrary, all heteroaryl groups described or claimed herein may be substituted or unsubstituted.

The term “heterocycloalkyl” unless otherwise specified refers to substituted or unsubstituted saturated 3 to 15 membered ring radical (i.e. 3 to 15 membered heterocycloalkyl) which consists of carbon atoms and from one to five hetero atoms selected from nitrogen, oxygen and sulfur. The heterocycloalkyl radical may be a mono-, bi- or tricyclic ring system, which may include fused, bridged or spiro ring systems, and the nitrogen or sulfur atoms in the heterocyclic ring radical may be optionally oxidized to various oxidation states. The heterocycloalkyl radical may be attached to the main structure at any heteroatom or carbon atom that results in the creation of a stable structure. Unless set forth or recited to the contrary, all heterocycloalkyl groups described or claimed herein may be substituted or unsubstituted. Examples of heterocycloalkyl include, but are not limited to aziridinyl, pyrrolidinyl, piperdinyl, piperazinyl, morpholinyl, 2-oxopyridyl, thiomorpholinyl, thiazolidinyl, 1,3-dioxolanyl, 1,4-dioxanyl, 1,1-dioxido-2,3-dihydrobenzo[b]thiophen-5-yl and the like.

As used herein, the term “heterocyclyl” alone or in combination with other term(s) refers to a saturated, partially saturated or unsaturated heterocyclic system. “Heterocyclyl” includes the definitions of both “heterocycloalkyl” and “heteroaryl” groups as well, which are as defined above. Heterocyclyl ring can be monocyclic; or one or more rings can be fused to form bicyclic or polycyclic heterocyclyl. Examples of “heterocyclyl” include, but are not limited to azetidinyl, pyrrolidinyl, piperidinyl, pyridyl, indolyl, benzimidazolyl, benzothiazolyl, pyridazinyl, imidazo[1,2-a]pyridinyl and the like.

As used herein, the term “optionally substituted” refers to replacement of one or more hydrogen radicals in a given structure with a radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

As used herein, the term “compound(s)” comprises the compounds disclosed in the present invention.

As used herein, the term “comprise” or “comprising” is generally used in the sense of include, that is to say permitting the presence of one or more features or components.

As used herein, the term “including” as well as other forms, such as “include”, “includes” and “included” is not limiting.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The term “pharmaceutically acceptable salt” includes salts prepared from pharmaceutically acceptable bases or acids including inorganic or organic bases and inorganic or organic acids. Examples of such salts include, but are not limited to, acetate, benzenesulfonate, benzoate, bicarbonate, bisulfate, tartrate, bitartrate, borate, bromide, camsylate, carbonate, citrate, clavulanate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, hydroiodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, teoclate, tosylate, triethiodide and valerate. Examples of salts derived from inorganic bases include, but are not limited to, aluminum, ammonium, calcium, copper, ferric, ferrous, lithium, magnesium, potassium, sodium and zinc.

As used herein, the term “pharmaceutically acceptable carrier” refers to any of the standard pharmaceutical carriers, such as a phosphate buffered saline solution, water, emulsions {e.g., such as an oil/water or water/oil emulsions), and various types of wetting agents. The compositions also can include stabilizers and preservatives, for example, carriers, stabilizers and adjuvants known in literature. By “pharmaceutically acceptable” it is meant the carrier, diluent or excipient must be compatible with the other ingredients of the formulation and not deleterious to the recipient thereof.

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; or (c) relieving the disease, i.e., causing regression of the state, disorder or condition or at least one of its clinical or subclinical symptoms.

The term “subject” includes mammals (especially humans) and other animals, such as domestic animals (e.g., household pets including cats and dogs) and non-domestic animals (such as wildlife).

As used herein, the term “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

As used herein, the term “composition” is intended to encompass a product comprising the specified ingredients in the specified amounts, as well as any product which results, directly or indirectly, from combination of the specified ingredients in the specified amounts.

The term “stereoisomers” refers to any enantiomers, diastereomers or geometrical isomers of the compounds of formula (I), wherever they are chiral or when they bear one or more double bond. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-isomers and l-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art.

Additionally, the compounds of the present invention may exist as geometric isomers. The present invention includes all cis, trans, syn, anti, entgegen (E) and zusammen (Z) isomers as well as the appropriate mixtures thereof.

The present invention provides 4,5-diydroisoxazole derivatives of formula (I), which are useful for the inhibition of NAMPT.

The present invention further provides pharmaceutical compositions comprising the said 4,5-diydroisoxazole derivatives as therapeutic agents.

The first embodiment of the present invention provides the compounds as set forth in formula (I):

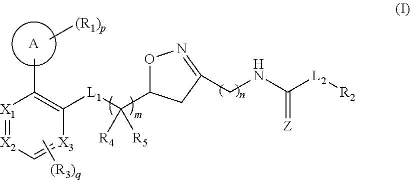

or a pharmaceutically acceptable salt or a stereoisomer thereof;
wherein,

ring A is carbocyclyl or heterocyclyl;

X₁, X₂ and X₃ independently are C or N; provided all X₁, X₂ and X₃ are not C at any instance;

L₁ is —O—, —S—, —SO—, —SO₂—, —NH—, —CO—, —NHCO—, —CONH—, —NHSO₂— or —SO₂NH—;

L₂ is a direct bond, —CH═CH— or —NR_{b}(CH₂)ᵣ—;

Z is O, S or NCN;

R₁ at each occurrence is independently alkyl, nitro, halo, haloalkyl, hydroxyalkyl, —ORₐ, —(CH₂)ₙNR_{b}R_{c}, —(SO₂)-alkyl, —(SO₂)—NR_{b}R_{c}, —NH(CO)alkyl or —(CO)NHR_{b};

R₂ is optionally substituted carbocyclyl or optionally substituted heterocyclyl wherein the optional substituent is R₆;

R₃ at each occurrence is hydrogen, halo, amino, nitro, cyano, alkyl, hydroxy, alkoxy, haloalkyl or haloalkoxy;

R₄ and R₅ independently are hydrogen or alkyl;

alternatively, R₄ and R₅ together with the carbon atom to which they are attached form an optionally substituted cycloalkyl ring; wherein the optional substituent is alkyl or halo;

R₆ at each occurrence is independently one or more alkyl, amino, halo, nitro, cyano, haloalkyl, hydroxyl, alkoxy, —NHSO₂-alkyl, cycloalkyl, optionally substituted aryl or optionally substituted heteroaryl wherein the optional substituent at each occurrence is halo, alkyl, hydroxyl, alkoxy or haloalkyl;

Rₐ is hydrogen, alkyl or arylalkyl;

R_{b} and R_{c} independently are hydrogen or alkyl;

m, n and p independently are 0, 1, 2 or 3;

q is 1, 2 or 3; and

r is 0, 1 or 2.

According to another embodiment the present invention provides compounds of the formula (IA),

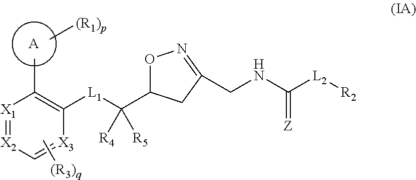

or a pharmaceutically acceptable salt or a stereoisomer thereof;

wherein, ring A, L₁, L₂, X₁, X₂, X₃, Z, R₁, R₂, R₃, R₄, R₅, p and q are same as defined in formula (I).

According to another embodiment, the present invention provides compounds of the formula (IB),

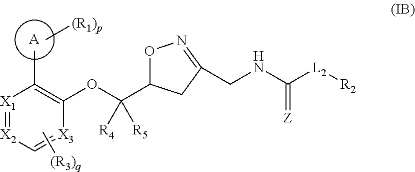

or a pharmaceutically acceptable salt or a stereoisomer thereof;

wherein, ring A, L₂, X₁, X₂, X₃, Z, R₁, R₂, R₃, R₄, R₅, p and q are same as defined in formula (I).

According to yet another embodiment, the present invention provides compounds of the formula (IB) wherein R₄ and R₅ are hydrogen.

According to another embodiment, the present invention provides compounds of the formula (IC),

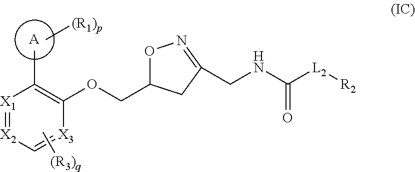

or a pharmaceutically acceptable salt or a stereoisomer thereof;

wherein, ring A, L₂, X₁, X₂, X₃, R₁, R₂, R₃, p and q are same as defined in formula (I).

According to another embodiment, the present invention provides compounds of the formula (ID),

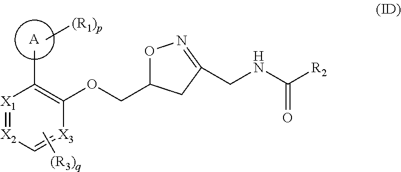

or a pharmaceutically acceptable salt or a stereoisomer thereof;

wherein, ring A, X₁, X₂, X₃, R₁, R₂, R₃, p and q are same as defined in formula (I).

According to another embodiment, the present invention provides compounds of the formula (IE),

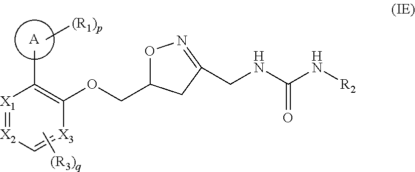

or a pharmaceutically acceptable salt or a stereoisomer thereof;

wherein, ring A, X₁, X₂, X₃, R₁, R₂, R₃, p and q are same as defined in formula (I).

According to yet another embodiment, the present invention provides compounds of the formula (IF),

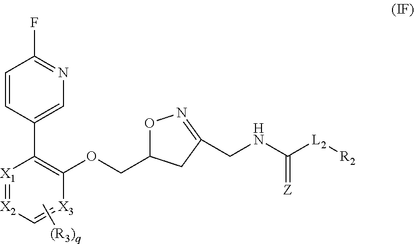

or a pharmaceutically acceptable salt or a stereoisomer thereof;

wherein, L₂, X₁, X₂, X₃, Z, R₂, R₃ and q are same as defined in formula (I).

According to yet another embodiment, the present invention provides compounds of the formula (IG),

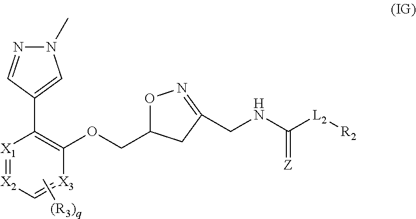

or a pharmaceutically acceptable salt or a stereoisomer thereof;

wherein, L₂, X₁, X₂, X₃, Z, R₂, R₃ and q are same as defined in formula (I).

According to yet another embodiment, the present invention provides compounds of the formula (IH),

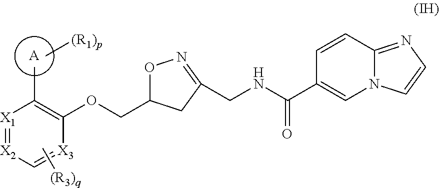

or a pharmaceutically acceptable salt or a stereoisomer thereof;

wherein, ring A, X₁, X₂, X₃, R₁, R₃, p and q are same as defined in formula (I).

According to yet another embodiment, the present invention provides compounds of the formula (I) in which

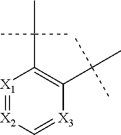

is

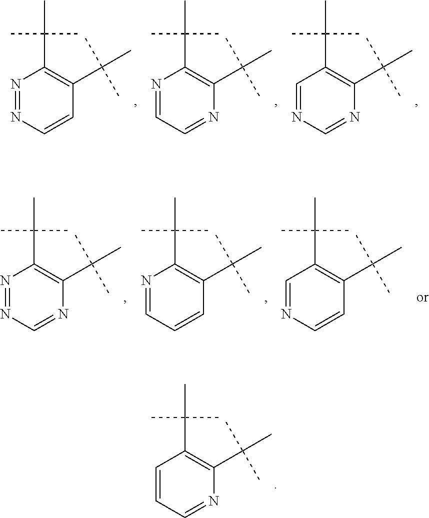

According to yet another embodiment, the present invention provides compounds of the formula (I) in which

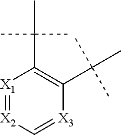

is pyridinyl.

The embodiments below are illustrative of the present invention and are not intended to limit the claims to the specific embodiments exemplified.

According to yet another embodiment, the present invention provides compounds of the formula (I) in which ring A is heteroaryl or aryl.

According to yet another embodiment, the present invention provides compounds of the formula (I) in which ring A is phenyl, pyridinyl or pyrazolyl.

According to yet another embodiment, the present invention provides compounds of the formula (I) in which Z is O or S; in one particular embodiment Z is O.

According to yet another embodiment, the present invention provides compounds of the formula (I) in which L₁ is —O— or —S—; in one particular embodiment L₁ is —O—.

According to yet another embodiment, the present invention provides compounds of the formula (I) in which L₂ is a direct bond or —NR_{b}(CH₂)ᵣ—; wherein R_{b} is H and r is 0.

According to yet another embodiment, the present invention provides compounds of the formula (I) in which R₁ at each occurrence is C₁-C₄ alkyl, halo or —(CO)NHR_{b}; wherein R_{b} is methyl.

According to the preceding embodiment, the said C₁-C₄ alkyl is methyl and the said halo is fluoro or chloro.

According to yet another embodiment, the present invention provides compounds of the formula (I) in which R₂ is optionally substituted heterocyclyl.

According to the preceding embodiment, the said optionally substituted heterocyclyl is pyridinyl, pyridazinyl or imidazo[1,2-a]pyridinyl wherein the optional substituent is methyl.

According to yet another embodiment, the present invention provides compounds of the formula (I) in which R₃ at each occurrence is halo, C₁-C₄ alkyl or cyano; wherein the said halo is fluoro or chloro and the said C₁-C₄ alkyl is methyl.

According to yet another embodiment, the present invention provides compounds of the formula (I) in which R₄ and R₅ independently are hydrogen or alkyl.

According to yet another embodiment, the present invention provides compounds of the formula (I) in which R₄ and R₅ both are hydrogen.

According to yet another embodiment, the present invention provides compounds of the formula (I) in which m and n are 1.

According to yet another embodiment, the present invention provides compounds of the formula (I) in which p is 0, 1 or 2.

According to yet another embodiment, the present invention provides compounds of the formula (I) in which q is 1, 2 or 3.

The present application also provides a pharmaceutical composition that includes at least one compound described herein and at least one pharmaceutically acceptable excipient (such as a pharmaceutically acceptable carrier or diluent). Preferably, the pharmaceutical composition comprises a therapeutically effective amount of at least one compound described herein. The compounds described in the present patent application may be associated with a pharmaceutically acceptable excipient (such as a carrier or a diluent) or be diluted by a carrier, or enclosed within a carrier which can be in the form of a capsule, sachet, paper or other container.

The compounds and pharmaceutical compositions of the present invention are useful for inhibiting the activity of NAMPT, which is believed to be related to a variety of disease states.

The present patent application further provides a method of inhibiting NAMPT in a subject in need thereof by administering to the subject one or more compounds described herein with an amount effective to cause inhibition of such receptor.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of this patent application to the appropriate or desired site of action.

The pharmaceutical composition(s) of the present invention can be administered orally, for example in the form of tablets, capsules (soft or hard gelatin), pills, granules, dragees (containing the active ingredient in powder or pellet form), troches and lozenges. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, impregnated dressings, or in other ways, for example in the form of aerosols, nasal sprays, eye or ear drops and may contain appropriate conventional additives such as preservatives, solvents to assist drug penetration.

Liquid formulations include, but are not limited to, syrups, emulsions, and sterile injectable liquids, such as suspensions or solutions.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present patent application.

It is contemplated that compounds disclosed in the present invention provide therapeutic benefits to subjects suffering from immune or inflammatory disorders and/or diseases. Accordingly, one embodiment of the present invention provides a method of treating disorders and/or diseases selected from the group consisting of immune or inflammatory disorder or diseases. The method comprises administering a therapeutically effective amount of a compound of the present invention, to a subject in need thereof to ameliorate a symptom of the disorder or diseases.

According to another embodiment, the disorder or disease is cancer.

According to another embodiment, the disorder or disease is an immune disorder or disease.

According to yet another embodiment, the disorder or disease is an inflammatory disorder or disease.

According to yet another embodiment, the disorder or disease is an autoimmune disorder or disease.

According to yet another embodiment, the disorders and/or diseases include, but are not limited to the group consisting of cancer, pancreatic cancer, ovarian cancer, lung cancer, prostate cancer, skin cancer, breast cancer, uterine cancer, renal cancer, head and neck cancer, brain cancer, colon cancer, cervical cancer, bladder cancer, leukemia, lymphoma, Hodgkin's disease, viral infections including adult respiratory distress syndrome, ataxia telengiectasia, Human Immunodeficiency Virus, hepatitis virus, herpes virus, herpes simplex, inflammatory disorders, irritable bowel syndrome, inflammatory bowel disease, rheumatoid arthritis, asthma, chronic obstructive pulmonary disease, osteoarthritis, osteoporosis, fibrotic diseases, dermatosis, atopic dermatitis, psoriasis, ultra-violet induced skin damage, systemic lupus erythematosis, multiple sclerosis, psoriatic arthritis, ankylosing spondylitis, graft-versus-host disease, Alzheimer's disease, cerebrovascular accident, atherosclerosis, restenosis, diabetes, glomerulonephiritis, metabolic syndrome, non-small cell lung cancer, small cell lung cancer, multiple myeloma, leukemias, lymphomas, cancers of the brain and central nervous system, squamous cell cancers, kidney cancer, uretral and bladder cancers, cancers of head and neck.

According to yet another embodiment, the disorder or disease is psoriasis.

According to yet another embodiment, the psoriasis is plaque psoriasis, guttate psoriasis, inverse psoriasis, pustular psoriasis or erythrodermic psoriasis.

According to yet another embodiment, the disorder or disease is rheumatoid arthritis.

According to yet another embodiment, the subject is a human.

According to yet another embodiment, the present invention provides compounds for use as a medicament.

According to yet another embodiment the invention provides use of the compounds of the present invention in the manufacture of a medicament.

According to yet another embodiment the invention provides use of the compounds of the present invention in the manufacture of a medicament for the treatment of immune or inflammatory disorder or disease.

According to yet another embodiment the present invention provides compounds for use as a medicament for the treatment of cancer.

According to yet another embodiment, the medicament is for treating a disease or disorder caused by an elevated level of nicotinamide phosphoribosyltransferase (NAMPT).

The method(s) of treatment of the present patent application comprise administering a safe and effective amount of a compound according to formula (I) or a pharmaceutically acceptable salt thereof or a stereoisomer thereof to a patient (particularly a human) in need thereof.

Compounds of the invention are indicated both in the therapeutic and/or prophylactic treatment of the above-mentioned conditions. For the above mentioned therapeutic uses the dosage administered will, of course, vary with the compound employed, the mode of administration, the treatment desired and the disorder or disease indicated.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, 18F, ³⁶Cl, ¹²³I and 125I. Isotopically labeled compounds of the present invention can generally be prepared by following procedures analogous to those disclosed in the schemes and/or in the examples herein below, by substituting an isotopically labeled reagent for a non-isotopically labeled reagent.

The MS (Mass Spectral) data provided in the examples were obtained using the following equipments.

API 2000 LC/MS/MS/Triplequad,

Agilent Technologies/LC/MS/DVL/Singlequad,

Shimadzu LCMS-2020/Singlequad.

The NMR data provided in the examples were obtained using the equipment—¹H

NMR: Varian 600 MHz, Varian 400 MHz and Varian 300 MHz

The HPLC performed for the provided examples using the equipments—

Agilent Technologies 1200 Series,

Agilent Technologies 1100 Series,

Shimadzu (UFLC) Prominence,

Shimadzu Nexera-UHPLC.

The following abbreviations refer respectively to the definitions below:

NCS—N-Chlorosuccinimide; DMF—N, N-Dimethylformamide; BOP Reagent—(Benzotriazol-1-yloxy)tris(dimethylamino)phosphonium hexafluorophosphate; DMSO—Dimethylsulfoxide; DIPEA—N,N-Diisopropylethylamine; HOBt—N-Hydroxybenzotriazole, Dioxane.HCl—Hydrochloric acid in dioxane; DEA-Diethyl amine; K₂CO₃—Potassium carbonate; Tetrakis—Tetrakis(triphenylphosphine)palladium(0); H₂O—water; ACN—Acetonitrile; K₃PO₄—Tripotassium phosphate; br—Broad; Pd(dppf)Cl₂—[1,1′-Bis(diphenylphosphino)ferrocene] dichloropalladium(II); PCy3—Tricyclohexylphosphine; Pd(dba)₃-Tris(dibenzylideneacetone)dipalladium(0); ° C.—Degree Celsius; Å—Angstrom; B(OH)₂—Boronic acid; cone—Concentrated; CDCl₃—Deuterated Chloroform; DMSO-d₆—Deuterated dimethylsulfoxide; CH₂Cl₂—DCM—Dichloromethane; g—Gram; h—Hours; ¹H—Proton; HCl—Hydrochloric acid; Hz—Hertz; J—Coupling Constant; LC-MS—Liquid Chromatography—Mass Spectroscopy; HPLC—High-performance liquid chromatography; chiral HPLC—chiral high-performance liquid chromatography; MeOH—methanol; M—Molar; MHz—Mega Hertz (frequency); MS—Mass Spectroscopy; mmol—milli mole; mL—Milli Litre; min—Minutes; mol—Moles; M⁺—Molecular ion; N—Normality; NMR—Nuclear Magnetic Resonance; Et₃N/TEA—Triethyl amine; ppm—Parts per million; rt/RT—Room temperature; s—Singlet; d—Doublet; t—Triplet; q—Quartet; m—Multiplet; dd—doublet of doublets; td—triplet of doublets; qd—quartet of doublets; ddd—doublet of doublet of doublets; dt—doublet of triplets; ddt—doublet of doublet of triplets; TLC—Thin Layer Chromatography; THF—Tetrahydrofuran; %—Percentage; μ—Micron; and δ—Delta; anh.—anhydrous; PMS—Phenazine methosulphate; XTT—2,3-Bis-(2-Methoxy-4-Nitro-5-Sulfophenyl)-2H-Tetrazolium-5-Carboxanilide; cDMEM—Dulbecco's Modified 15 Eagle's Medium; FBS—Fetal bovine serum; NAM—Nam—Nicotinamide; NMN—Nmn—Nicotinamide mononucleotide; nm—nanometer; PRPP—Phosphoribosyl pyrophosphate; ATP—Adenosine triphosphate; BSA—Bovine serum albumin; DTT—Dithiothreitol; MgCl₂—Magnesium chloride; IC50—Inhibitory concentration 50; RFU—relative fluorescence units and Tris—tris(hydroxymethyl)aminomethane.

### General Modes of Preparation:

Methods for preparing compounds described herein are illustrated in the following examples. The schemes are given for the purpose of illustrating the invention, and are not intended to limit the scope or spirit of the invention. Starting materials shown in the schemes can be obtained from commercial sources or prepared based on procedures described in the literature. Furthermore, in the following schemes, where specific acids, bases, reagents, coupling agents, solvents, etc. are mentioned, it is understood that other suitable acids, bases, reagents, coupling agents etc. may be used and are included within the scope of the present invention. Modifications to reaction conditions, for example, temperature, duration of the reaction or combinations thereof, are envisioned as part of the present invention. All possible stereoisomers are envisioned within the scope of this invention.

The intermediates required for the synthesis are commercially available or alternatively, these intermediates can be prepared using known literature methods. The invention is described in greater detail by way of specific examples.

It is contemplated that some of the intermediates disclosed in the present invention are used for the next step without any characterization data.

It is meant to be understood that the order of the steps in the processes may be varied, that reagents, solvents and reaction conditions may be substituted for those specifically mentioned, and that vulnerable moieties may be protected and deprotected, as necessary.

### General Synthetic Schemes

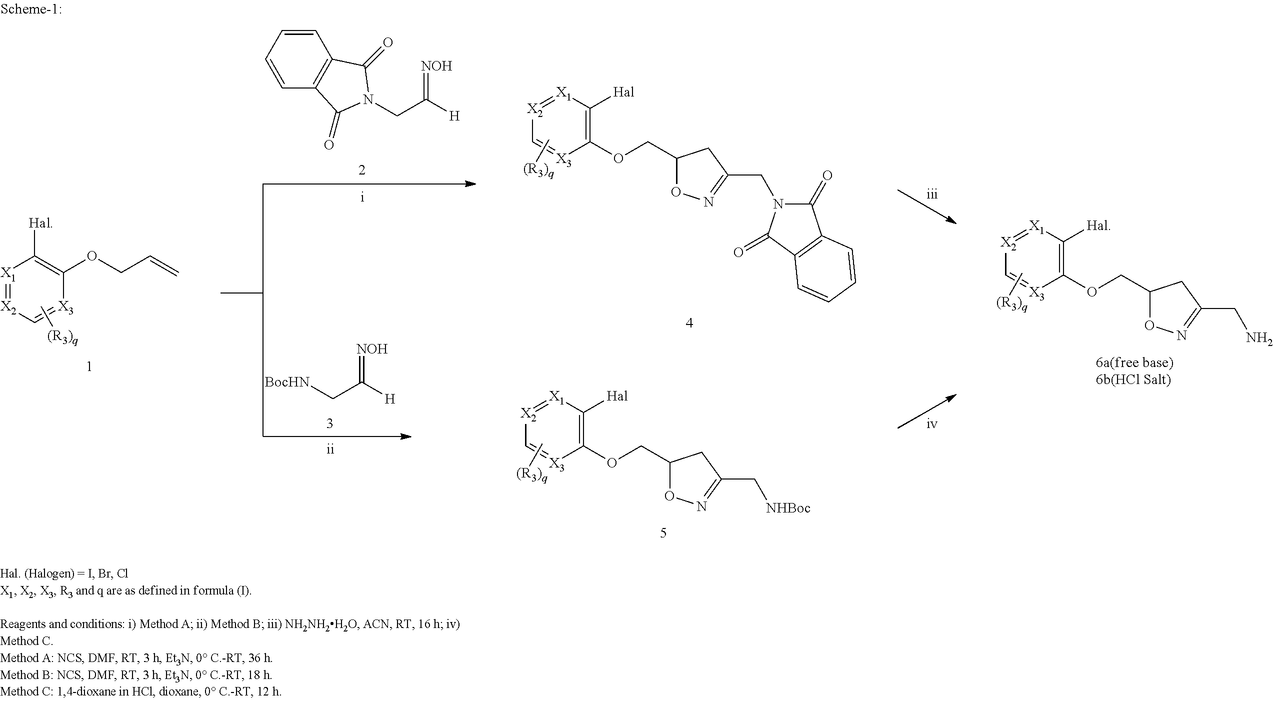

Intermediates of formula 5, 6a and 6b of the present invention may be synthesized using the process outlined in general synthetic scheme-1. The commercially available or synthesized intermediate of formula 1 upon treating with intermediate of formula 2 in presence of a suitable solvent (DMF) and N-chlorosuccinimide under suitable conditions gives intermediate of formula 4, which upon treating with hydrazine hydrate under suitable conditions gives intermediate of formula 6a.

Alternatively, intermediate of formula 1 upon treating with intermediate of formula 3 in presence of a suitable solvent (DMF) and N-chlorosuccinimide under suitable conditions gives intermediate of formula 5, which upon treating with 1,4-dioxane in HCl yields intermediate of formula 6b.

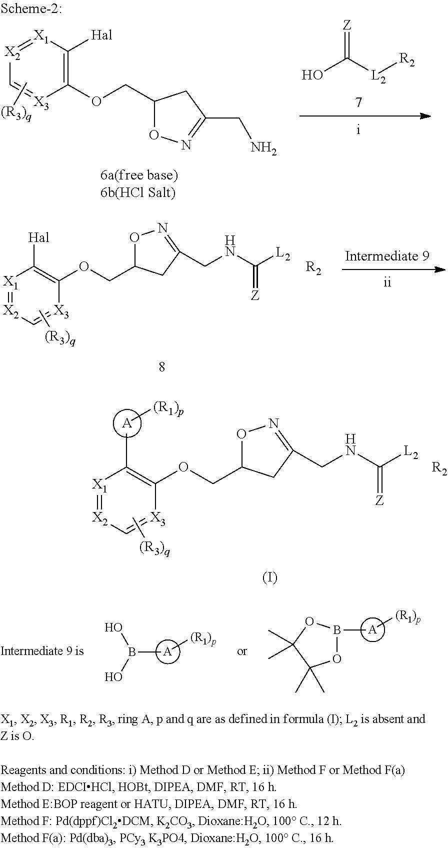

The first general approach for the synthesis of compounds of general formula (I) is depicted in general synthetic scheme-2. The intermediate of formula 6a/6b upon treating with intermediate of formula 7 in presence of a suitable base (DIPEA) and suitable solvent (DMF) under suitable conditions gives intermediate of formula 8 which upon treating with intermediate of formula 9 in presence of a suitable base (K₂CO₃) and suitable solvent (Dioxane:water) under suitable coupling conditions afford the desired compound of formula (I).

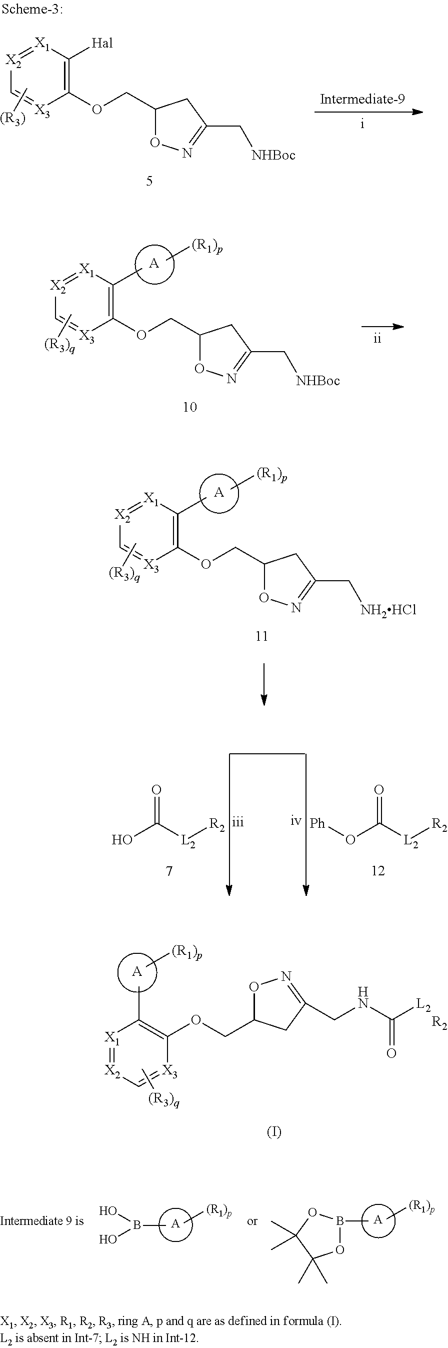

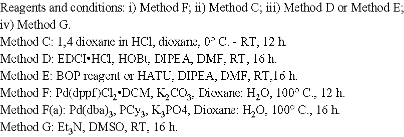

Another general approach for the synthesis of compounds of formula (I) is depicted in general synthetic scheme-3. Intermediate of formula-5 upon reacting with intermediate of formula-9 in presence of a suitable base (K₂CO₃) and suitable solvent (Dioxane:water) under suitable conditions gives intermediate of formula-10. Intermediate of formula-10 upon deprotection under suitable conditions (HCl in Dioxane) gives intermediate of formula-11, which upon coupling with intermediate of formula-7 in presence of a suitable base (DIPEA) and suitable solvent (DMF) under suitable conditions affords the desired compound of formula (I).

Alternatively, Intermediate of formula 11 upon treating with intermediate of formula 12 in presence of a suitable base (Et₃N) and a suitable solvent (DMSO) under suitable conditions affords the desired compound of formula (I).

The specifics of the process for preparing compounds of the present invention are detailed in the experimental section.

The present invention shall be illustrated by means of some examples, which are not construed to be viewed as limiting the scope of the invention.

### EXPERIMENTAL

Unless otherwise stated, work-up includes distribution of the reaction mixture between the organic and aqueous phase indicated, separation of layers and drying the organic layer over anhydrous sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase. Use of a different eluent system is indicated as applicable.

Analysis for the compounds of the present invention unless mentioned, was conducted in the general methods well known to the person skilled in the art. Having described the invention with reference to certain preferred embodiments, other embodiments will become apparent to one skilled in the art from consideration of the specification. The invention is further defined by reference to the following examples, describing in detail the analysis of the compounds of the invention.

It will be apparent to those skilled in the art that many modifications, both to materials and methods, may be practiced without departing from the scope of the invention.

### EXAMPLES

The present invention is further exemplified, but not limited, by the following examples that illustrate the preparation of compounds according to the invention.

### Example-1: N-((5-(((3-(1-methyl-1H-pyrazol-4-yl)pyridin-2-yl)oxy)methyl)-4,5-dihydroisoxazol-3-yl)methyl)imidazo[1,2-a]pyridine-6-carboxamide (±)

### Step-1: 2-(allyloxy)-3-bromopyridine

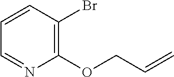

To a solution of 3-bromo-2-fluoropyridine (1.0 g, 5.68 mmol) and allyl alcohol (1.96 mL, 5.0 mmol) in dry THF (10 mL), was added sodium hydride (0.55 g, 2.4 mmol, 60%) portion wise. The reaction mixture was stirred at room temperature for 16 h. The reaction mixture was quenched by adding water (50 mL), extracted with ethyl acetate (2×100 mL).

The combined organic layer was dried over sodium sulphate and evaporated under reduced pressure to afford the title compound (1.4 g, 98%) as oil. The crude product was taken to next step without further purification. LCMS: m/z 214 [M+2]⁺; ¹H NMR (300 MHz, Chloroform-d) δ 8.13-8.00 (dd, J=4.8, 1.8 Hz, 1H), 7.88-7.72 (dd, J=7.6, 1.7 Hz, 1H), 6.88-6.68 (dd, J=7.6, 4.9 Hz, 1H), 6.22-5.97 (ddt, J=17.4, 10.5, 5.2, 5.2 Hz, 1H), 5.53-5.37 (dq, J=17.2, 1.7, 1.6, 1.6 Hz, 1H), 5.35-5.20 (dq, J=10.4, 1.5, 1.4, 1.4 Hz, 1H), 4.99-4.82 (dt, J=5.0, 1.7, 1.7 Hz, 2H).

### Step-2: 2-(2,2-dimethoxyethyl) isoindoline-1,3-dione

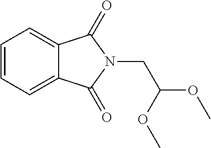 [SMILES: COC(CN1C(=O)c2ccccc2C1=O)OC]

To a solution of isobenzofuran-1,3-dione (80.0 g, 0.540 mol) in toluene (1000 mL), added 2,2-dimethoxyethanamine (85.10 g, 0.811 mol) followed by N,N-diisopropylethylamine (0.187 mL, 1.080 mol) at room temperature. The reaction mixture was stirred at 120° C. with dean stark apparatus for 16 h. After completion of the reaction, the reaction mixture was evaporated under reduced pressure. The residue obtained was diluted with dichloromethane, filtered through pad of Celite® and dried over sodium sulphate and concentrated. The crude obtained was washed with petroleum ether to get the title compound (100 g, 79%) as an off white solid. LCMS: m/z no ionization; ¹H NMR (300 MHz, Chloroform-d) δ 7.89-7.81 (m, 2H), 7.76-7.68 (m, 2H), 4.77 (td, J=5.8, 0.8 Hz, 1H), 3.82 (dd, J=5.8, 0.8 Hz, 2H), 3.38 (d, J=0.9 Hz, 6H).

### Step-3: 2-(1,3-dioxoisoindolin-2-yl)acetaldehyde

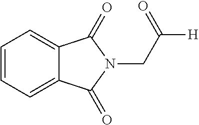 [SMILES: O=CCN1C(=O)c2ccccc2C1=O]

The solution of 2-(2,2-dimethoxyethyl)isoindoline-1,3-dione (product of step-2, 100 g, 0.425 mol) in 1N HCl (100 mL) was stirred at 80° C. for 2 h. The reaction mixture was cooled to room temperature, diluted with water (200 mL) and extracted with ethyl acetate (2×200 mL). The combined organic layer was washed with water (2×200 mL) followed by brine, dried over sodium sulphate and concentrated under vacuum to afford the title compound (60.0 g, 75%) as an off white solid. LCMS: m/z 190.1 [M+H]⁺; ¹H NMR (300 MHz, Chloroform-d) δ 9.66 (d, J=0.8 Hz, 1H), 7.92-7.88 (m, 2H), 7.79-7.74 (m, 2H), 4.57 (d, J=0.8 Hz, 2H).

### Step-4: 2-(1,3-dioxoisoindolin-2-yl)acetaldehyde oxime

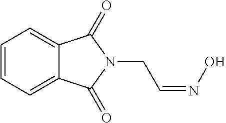 [SMILES: O=C1c2ccccc2C(=O)N1C/C=N\O]

To a stirred solution of 2-(1,3-dioxoisoindolin-2-yl)acetaldehyde (product of step-3, 4.0 g, 21.15 mmol) in ethanol (40 mL), added hydroxylamine hydrochloride (2.930 g, 42.305 mmol) followed by sodium bicarbonate (3.55 g, 42.305 mmol) at room temperature and stirred at room temperature for 16 h. The solvent was stripped off, added water (50 mL) and extracted with ethyl acetate (2×100 mL). The combined organic layer was washed with water (100 mL) followed by brine, dried over sodium sulphate and concentrated under vacuum to get the title compound (3.2 g, 74%) as an off white solid. LCMS: m/z 204.1 [M+H]⁺; ¹H NMR (300 MHz, DMSO-d6) δ 11.36 (s, 1H), 7.98-7.78 (m, 4H), 6.83 (t, J=3.8 Hz, 1H), 4.39 (dd, J=3.9, 0.9 Hz, 2H).

### Step-5: 2-((5-(((3-bromopyridin-2-yl)oxy)methyl)-4,5-dihydroisoxazol-3-yl)methyl)isoindoline-1,3-dione (±)

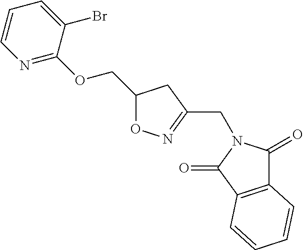 [SMILES: O=C1c2ccccc2C(=O)N1CC1=NOC(COc2ncccc2Br)C1]

To a solution of 2-(1,3-dioxoisoindolin-2-yl)acetaldehyde oxime (product of step-4, 1.3 g, 6.54 mmol) in DMF (20 mL) was added N-chlorosuccinimide (0.96 g, 7.2 mmol) at room temperature and stirred for 3 h, then cooled to 0° C. and added 2-(allyloxy)-3-bromopyridine (product of step-1, 1.4 g, 6.54 mmol) in one lot followed by drop wise addition of solution of triethylamine (1.0 mL, 7.2 mmol) in DMF (5 mL) over 10 min. The reaction mixture was stirred further for 36 h at room temperature. The reaction mixture was poured onto water, extracted with ethyl acetate (50 mL) and ethyl acetate layer was washed with water (4×50 mL). The organic phase was dried over sodium sulphate and concentrated under reduced pressure to afford a residue. The residue was purified by column chromatography on silica gel (hexanes/ethyl acetate=80/20) to get the title compound (0.400 g, 15%) as a solid. LCMS: m/z 417.6 [M+H]⁺; ¹H NMR (300 MHz, Chloroform-d) 8.08-7.99 (dd, J=4.9, 1.7 Hz, 1H), 7.94-7.83 (dd, J=5.5, 3.1 Hz, 2H), 7.83-7.69 (ddt, J=7.1, 5.5, 2.4, 2.4 Hz, 3H), 6.82-6.71 (dd, J=7.6, 4.9 Hz, 1H), 5.11-4.94 (m, 1H), 4.53-4.33 (qd, J=11.5, 11.5, 11.5, 4.5 Hz, 3H), 3.28-3.00 (m, 3H).

### Step-6: (5-(((3-bromopyridin-2-yl)oxy)methyl)-4,5-dihydroisoxazol-3-yl)methanamine (±)

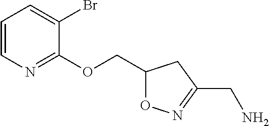 [SMILES: NCC1=NOC(COc2ncccc2Br)C1]

A suspension of 2-((5-(((3-bromopyridin-2-yl)oxy)methyl)-4,5-dihydroisoxazol-3-yl)methyl) isoindoline-1,3-dione (±) (product of step-5, 0.400 g, 0.960 mmol) in hydrazine hydrate (2 mL) was stirred at room temperature for 16 h. Acetonitrile (50 mL) was added to the reaction mixture and resulting precipitate was filtered. The filtrate was evaporated under reduced pressure to afford the title compound (0.070 g, 25%). The crude product was taken to the next step without further purification. LCMS: m/z 287.9 [M+2]⁺.

### Step-7: N-((5-(((3-bromopyridin-2-yl)oxy)methyl)-4,5-dihydroisoxazol-3-yl)methyl)imidazo-[1,2-a]pyridine-6-carboxamide (±)

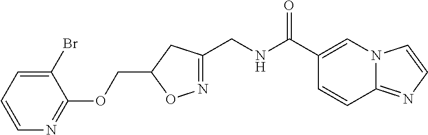 [SMILES: O=C(NCC1=NOC(COc2ncccc2Br)C1)c1ccc2nccn2c1]

To a solution of (5-(((3-bromopyridin-2-yl)oxy)methyl)-4,5-dihydroisoxazol-3-yl)methanamine (±) (product of step-6, 0.07 g, 0.245 mmol) and imidazo[1,2-a]pyridine-6-carboxylic acid (0.06 g, 0.367 mmol) in DMF (2 mL), added EDCI (0.094 g, 0.49 mmol), HOBT (0.066 g, 0.49 mmol) and DIPEA (0.17 mL, 0.98 mmol) at 0° C. The reaction mixture was stirred for 16 h at room temperature. The reaction mixture was diluted with ethyl acetate (250 mL). The organic phase was washed with aqueous sodium bicarbonate solution (50 mL), brine (3×50 mL), dried over sodium sulphate and concentrated in vacuum to afford a residue. The residue was purified by column chromatography on silica gel (dichloromethane/methanol=96/4) to afford the title compound (0.07 g, 66%) as a sticky solid. LCMS: m/z 431.40 [M+2]⁺.

### Step-8: N-((5-(((3-(1-methyl-1H-pyrazol-4-yl)pyridin-2-yl)oxy)methyl)-4,5-dihydroisoxazol-3-yl)methyl)imidazo[1,2-a]pyridine-6-carboxamide (±)

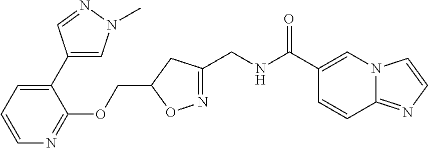 [SMILES: Cn1cc(-c2cccnc2OCC2CC(CNC(=O)c3ccc4nccn4c3)=NO2)cn1]

To a previously degassed solution of N-((5-(((3-bromopyridin-2-yl)oxy)methyl)-4,5-dihydroisoxazol-3-yl)methyl)imidazo[1,2-a]pyridine-6-carboxamide (±) (product of step-7, 0.070 g, 0.163 mmol) in dioxane:water (3:1) (3 mL), was added 1-methyl-4-(4,4,5,5-tetramethyl-1,3-dioxolan-2-yl)-1H-pyrazole (0.050 g, 0.244 mmol) followed by potassium carbonate (0.067 g, 0.489 mmol) and [1,1′-bis(diphenylphosphino)ferrocene]-dichloropalladium(II) complex with dichloromethane (0.013 g, 0.016 mmol) at room temperature under nitrogen atmosphere. The resulting reaction mixture was stirred for 12 h at 100° C. The reaction mixture was diluted with water (10 mL) extracted with ethyl acetate (50 mL) and ethyl acetate layer was washed with water (4×50 mL). The organic phase was dried over sodium sulphate and concentrated under reduced pressure. The residue was purified by column chromatography on silica gel (dichloromethane/methanol=96/4) to get the title compound (0.035 g, 50%) as a solid. LCMS: m/z 433.0 [M+H]⁺; HPLC: 94.02%; ¹H NMR (600 MHz, cdcl3) δ 8.82-8.75 (d, J=1.7 Hz, 1H), 8.00-7.94 (dd, J=5.0, 1.8 Hz, 1H), 7.93 (s, 1H), 7.83 (s, 1H), 7.73-7.65 (m, 2H), 7.65 (s, 1H), 7.61-7.56 (d, J=9.4 Hz, 1H), 7.46-7.37 (dd, J=9.4, 1.7 Hz, 1H), 7.09-6.98 (q, J=7.7, 7.7, 5.7 Hz, 1H), 6.94-6.84 (dd, J=7.4, 4.9 Hz, 1H), 5.15-5.04 (ddt, J=10.5, 6.9, 3.4, 3.4 Hz, 1H), 4.62-4.53 (dd, J=11.8, 2.8 Hz, 1H), 4.53-4.45 (dd, J=11.8, 3.8 Hz, 1H), 4.43-4.27 (qd, J=16.9, 16.9, 16.8, 5.6 Hz, 2H), 3.96 (s, 3H) 3.27-3.13 (dd, J=17.4, 11.4 Hz, 1H), 3.06-2.93 (dd, J=17.4, 6.8 Hz, 1H).

### Example-2: N-((5-(((6′-fluoro-[3,3′-bipyridin]-2-yl)oxy)methyl)-4,5-dihydroisoxazol-3-yl)methyl)-imidazo[1,2-a]pyridine-6-carboxamide (±)

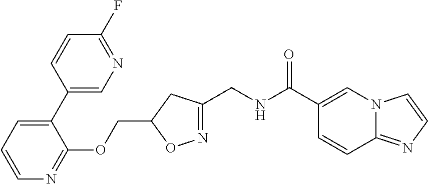 [SMILES: O=C(NCC1=NOC(COc2ncccc2-c2ccc(F)nc2)C1)c1ccc2nccn2c1]

N-((5-(((3-bromopyridin-2-yl)oxy)methyl)-4,5-dihydroisoxazol-3-yl)methyl)imidazo-[1,2-a]pyridine-6-carboxamide (±) (product of step-7 of example-1, 0.420 g, 0.980 mmol) was treated with (6-fluoropyridin-3-yl)boronic acid (0.207 g, 1.460 mmol) in 1,4 dioxane:water (3:1) (30 mL) as described in step-8 of example-1. The crude product obtained was purified by preparative HPLC to get the title compound (0.191 g, 43% W/W) as white solid. LCMS: m/z 447.1 [M+H]⁺; HPLC: 94.33%; ¹H NMR (400 MHz, Chloroform-d) δ 9.06-8.90 (d, J=1.7 Hz, 1H), 8.63-8.44 (d, J=2.5 Hz, 1H), 8.31-8.07 (m, 2H), 8.05-7.88 (td, J=8.0, 7.5, 2.6 Hz, 1H), 7.82-7.60 (m, 5H), 7.15-6.97 (m, 2H), 5.15-4.98 (m, 1H), 4.76-4.61 (dd, J=12.0, 2.6 Hz, 1H), 4.52-4.41 (dd, J=16.9, 5.8 Hz, 1H), 4.39-4.25 (m, 2H), 3.35-3.19 (m, 1H), 3.10-2.99 (dd, J=17.4, 6.6 Hz, 1H). Preparative HPLC Method: Column: Zorbax Eclipse XDB C18 (21.2×250 mm 5 μm); 0.1% TFA in Water (A) and Acetonitrile (B); Isocratic: 90:10.

### Example-3: N-((5-(((6′-fluoro-[3,3′-bipyridin]-4-yl)oxy)methyl)-4,5-dihydroisoxazol-3-yl)methyl)-imidazo[1,2-a]pyridine-6-carboxamide (±)

### Step-1: 4-(allyloxy)-3-iodopyridine

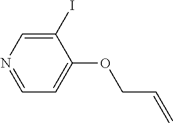

3-Iodo-4-hydroxypyridine (0.500 g, 2.250 mmol) was reacted with allyl bromide (0.409 g, 3.380 mmol), potassium carbonate (0.780 g, 5.630 mmol) and potassium iodide (0.016 g, 0.100 mmol) in acetone (20 mL) at reflux temperature for 2 h. Then reaction mixture was cooled to room temperature, filtered and the filtrate was concentrated under reduced pressure. The residue was purified by column chromatography on silica gel (hexanes/ethyl acetate=90/10) to get the title compound (0.400 g, 67%) as a liquid. LCMS: m/z 261.9 [M+1]⁺; ¹H NMR (300 MHz, DMSO-d₆) δ 7.86-7.85 (d, J=2.4 Hz, 1H), 7.31-7.28 (m, 1H), 6.41-6.38 (d, J=7.2 Hz, 1H), 5.97-5.86 (m, 1H), 5.43-5.40 (dd, J=9.6, 1.5 Hz, 1H), 5.29-5.25 (dd, J=10.8, 1.0 Hz, 1H), 4.40-4.38 (m, 2H).

### Step-2: Tert-butyl (2-(hydroxyimino)ethyl)carbamate (mixture of E and Z isomer)

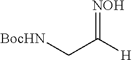

Tert-butyl(2-oxoethyl)carbamate (18.5 g, 116 mmol), hydroxylamine hydrochloride (13.8 g, 232 mmol) and sodium bicarbonate (24.3 g, 232 mmol) were dissolved in ethanol (200 rnL) and the reaction was stirred for 12 h at room temperature. Solvent was distilled under reduced pressure. The reaction mixture was diluted with ethyl acetate (100 mL) and washed with water (2×100 mL). The organic phase was dried over anhydrous sodium sulphate and concentrated under reduced pressure, afforded the title compound (18.5 g, crude) as a liquid (mixture of E and Z isomer). The crude product was taken to the next step without any further purification.

### Step-3: Tert-butyl ((5-(((3-iodopyridin-4-yl)oxy)methyl)-4,5-dihydroisoxazol-3-yl)methyl) carbamate (±)

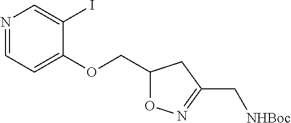 [SMILES: CC(C)(C)OC(=O)NCC1=NOC(COc2ccncc2I)C1]

Tert-butyl(2-(hydroxyimino)ethyl)carbamate (product of step-2, 0.300 g, 1.724 mmol) was reacted with N-chlorosuccinimide (0.346 g, 2.586 mmol) and then with 4-(allyloxy)-3-iodopyridine (product of step-1, 0.400 g, 1.550 mmol) in DMF (20 mL) as described in step-5 of example-1. The residue was purified by column chromatography on silica gel (hexanes/ethyl acetate=50/50) to get the title compound (0.200 g, 27%) as a liquid. LCMS: m/z 434.0 [M+H]; ¹HNMR (300 MHz, Chloroform-d) δ 7.99-7.86 (d, J=2.2 Hz, 1H), 7.46-7.31 (d, J=7.1 Hz, 1H), 6.44-6.31 (d, J=7.4 Hz, 1H), 5.06-4.78 (m, 2H), 4.19-3.76 (m, 4H), 3.33-3.14 (m, 1H), 2.86-2.65 (dd, J=17.5, 6.8 Hz, 1H), 1.56 (s, 9H).

### Step-4: Tert-butyl ((5-(((6′-fluoro-[3,3′-bipyridin]-4-yl)oxy)methyl)-4,5-dihydroisoxazol-3-yl)methyl)carbamate (±)

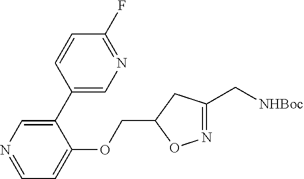 [SMILES: CC(C)(C)OC(=O)NCC1=NOC(COc2ccncc2-c2ccc(F)nc2)C1]

To a degassed solution of tert-butyl((5-(((3-iodopyridin-4-yl)oxy)methyl)-4,5-dihydroisoxazol-3-yl)methyl)carbamate (±) (product of step-3, 0.200 g, 0.462 mmol) in 1,4-dioxane:water (9:1) (20 mL), was added (6-fluoropyridin-3-yl)boronic acid (0.078 g, 0.554 mmol) followed by potassium carbonate (0.160 g, 1.150 mmol) and [1,1′-Bis(diphenylphosphino)ferrocene]-dichloropalladium(II) complex with dichloromethane (0.038 g, 0.046 mmol) at room temperature under inert atmosphere. Then resulting reaction mixture was stirred for 16 h at 90° C. The reaction mixture was cooled to room temperature, diluted with water (50 mL) and extracted with ethyl acetate (2×50 mL). The organic layer was separated and dried over anhydrous sodium sulphate, concentrated under reduced pressure. The residue obtained was purified by column chromatography on neutral alumina (dichloromethane/methanol=90/5) to get the title compound (0.100 g, 54%) as an off white solid. LCMS: m/z 403.1[M+H]⁺; ¹HNMR (400 MHz, Chloroform-d) δ 8.37 (s, 1H), 8.27-8.13 (t, J=8.0, 8.0 Hz, 1H), 7.71-(s, 1H), 7.51-7.41 (d, J=7.1 Hz, 1H), 6.99-6.86 (d, J=8.4 Hz, 1H), 6.57-6.46 (d, J=7.5 Hz, 1H), 5.09-4.87 (m, 2H), 4.29-3.86 (m, 4H), 3.35-3.16 (dd, J=17.6, 10.7 Hz, 1H), 2.87-2.71 (dd, J=17.5, 6.6 Hz, 1H), 2.01 (s, 9H).

### Step-5: (5-(((6′-fluoro-[3,3′-bipyridin]-4-yl)oxy)methyl)-4,5-dihydroisoxazol-3-yl)methanamine hydrochloride (±)

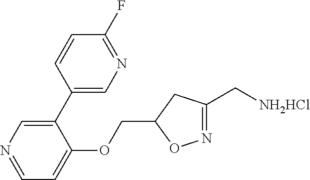

To a solution of Tert-butyl((5-(((6′-fluoro-[3,3′-bipyridin]-4-yl)oxy)methyl)-4,5-dihydroisoxazol-3-yl)methyl) carbamate (±) (product of step-4, 0.100 g, 2.487 mmol) in 1,4-dioxane (10 mL), drop wise added 1,4-dioxane.HCl (5 mL) at 0° C. The reaction mixture was stirred for 12 h at room temperature followed by concentration under reduced pressure. The solid obtained was washed with dry diethyl ether (2×10 mL) to get title compound (0.060 g, crude) as a solid. LCMS: m/z 303.1 [M+H]⁺.

### Step-6: N-((5-(((6′-fluoro-[3,3′-bipyridin]-4-yl)oxy)methyl)-4,5-dihydroisoxazol-3-yl)methyl) imidazo[1,2-a]pyridine-6-carboxamide (±)

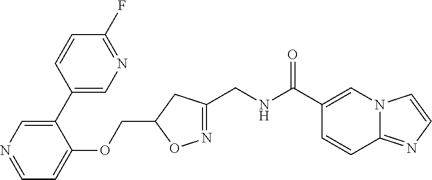 [SMILES: O=C(NCC1=NOC(COc2ccncc2-c2ccc(F)nc2)C1)c1ccc2nccn2c1]

(5-(((6′-Fluoro-[3,3′-bipyridin]-4-yl)oxy)methyl)-4,5-dihydroisoxazol-3-yl)methanamine hydrochloride (±) (product of step-5, 0.060 g, 0.198 mmol) was reacted with imidazo[1,2-a]pyridine-6-carboxylic acid (0.048 g, 0.297 mmol) in presence of HATU (0.113 g, 0.297 mmol) and N,N-diisopropylethylamine (0.100 mL, 0.495 mmol) in DMF (10 mL) as described in the synthesis of step-7 of example-1 to get the title compound (0.005 g, 6%) as a solid. LCMS: m/z 447.1 [M+H]; HPLC: 87.92%; ¹HNMR (400 MHz, Methanol-d₄) δ 9.24 (s, 1H), 8.37-8.23 (m, 2H), 8.23-8.02 (m, 4H), 7.96-7.81 (m, 2H), 7.11-7.00 (dd, J=8.7, 2.7 Hz, 1H), 6.57-6.47 (d, J=7.4 Hz, 1H), 5.14 (s, 1H), 4.43-4.25 (m, 2H), 4.20-4.04 (dd, J=14.5, 6.1 Hz, 1H), 3.47-3.10 (m, 2H), 3.07-2.91 (m, 1H).

### Example-4: N-((5-(((6′-fluoro-[2,3′-bipyridin]-3-yl)oxy)methyl)-4,5-dihydroisoxazol-3-yl)methyl)imidazo[1,2-a]pyridine-6-carboxamide (±)

### Step-1: 3-(allyloxy)-2-bromopyridine

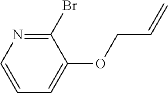

2-Bromo-3-hydroxypyridine (2.0 g, 11.494 mmol) was reacted with allyl bromide (2.08 g, 17.241 mmol), potassium carbonate (3.97 g, 28.730 mmol) and potassium iodide (0.016 g, 0.100 mmol) in acetone (50 mL) at reflux temperature for 2 h. The reaction mixture was filtered at room temperature; filtrate was concentrated under reduced pressure. The residue obtained was purified by column chromatography on silica gel column (hexanes/ethyl acetate=90/10) to get the title compound (1.80 g, 74%) as a liquid. LCMS: m/z 216.0 [M+2]+; ¹HNMR (400 MHz, Chloroform-d) δ 8.04-7.93 (dd, J=4.6, 1.6 Hz, 1H), 7.34-7.03 (m, 2H), 6.17-5.87 (ddt, J=17.2, 10.3, 5.0, 5.0 Hz, 1H), 5.61-5.40 (dq, J=17.2, 1.6, 1.6, 1.6 Hz, 1H), 5.40-5.26 (dq, J=10.7, 1.5, 1.5, 1.5 Hz, 1H), 4.69-4.44 (dt, J=4.8, 1.6, 1.6 Hz, 2H).

### Step-2: Tert-butyl ((5-(((2-bromopyridin-3-yl)oxy)methyl)-4,5-dihydroisoxazol-3-yl)methyl) carbamate (±)

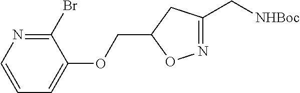 [SMILES: CC(C)(C)OC(=O)NCC1=NOC(COc2cccnc2Br)C1]

To a solution of tert-butyl (2-(hydroxyimino)ethyl)carbamate (product of step-2 of example-3, 1.770 g, 10.190 mmol) in DMF (50 mL), was added NCS (1.710 g, 12.740 mmol) at room temperature. The reaction mixture was stirred at room temperature for 3 h then cooled to 0° C. and added 3-(allyloxy)-2-bromopyridine (product of step-1, 1.80 g, 8.490 mmol) in one lot followed by drop wise addition of triethylamine (3.0 mL, 21.230 mmol). The reaction mixture was stirred further for 18 h at room temperature. The reaction mixture was poured onto ice water, extracted with ethyl acetate (2×50 mL). The organic phase was dried over sodium sulphate and concentrated under reduced pressure. The residue obtained was purified by column chromatography on silica gel column (hexanes/ethyl acetate=50/50) to get the title compound (1.20 g, 49.5%) as a liquid. LCMS: m/z 285.9 [M-100]+; ¹HNMR (400 MHz, Chloroform-d) δ 8.08-8.01 (m, 1H), 7.25-7.22 (m, 1H), 7.20-7.16 (m, 1H), 5.82-5.62 (m, 2H), 5.56-5.37 (m, 2H), 4.54-4.38 (m, 2H), 4.13-4.03 (m, 1H), 3.36-3.26 (m, 1H), 1.71-1.57 (m, 9H).

### Step-3: (5-(((2-bromopyridin-3-yl)oxy)methyl)-4,5-dihydroisoxazol-3-yl)methanamine hydrochloride (±)

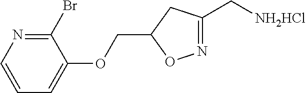

Tert-butyl((5-(((2-bromopyridin-3-yl)oxy)methyl)-4,5-dihydroisoxazol-3-yl)methyl)-carbamate (±) (product of step-2, 1.20 g, 3.10 mmol) in 1,4-dioxane (10 mL) drop wise added 1,4-dioxane.HCl (15 mL) at 0° C. The reaction mixture was stirred for 12 h at room temperature, concentrated under reduced pressure to get crude solid. This solid was washed with dry diethyl ether (2×10 mL) to afford the title compound (0.900 g, crude) as a solid. LCMS: m/z (no ionization observed). The product obtained was taken to next step.

### Step-4: N-((5-(((2-bromopyridin-3-yl)oxy)methyl)-4,5-dihydroisoxazol-3-yl)methyl)imidazo-[1,2-a]pyridine-6-carboxamide (±)

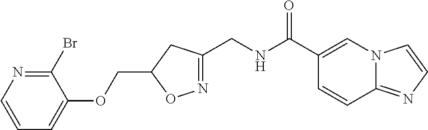 [SMILES: O=C(NCC1=NOC(COc2cccnc2Br)C1)c1ccc2nccn2c1]

(5-(((2-Bromopyridin-3-yl)oxy)methyl)-4,5-dihydroisoxazol-3-yl)methanamine hydrochloride (±) (product of step-3, 0.900 g, 2.800 mmol) was reacted with imidazo[1,2-a]pyridine-6-carboxylic acid (0.544 g, 3.350 mmol) in presence of HATU (1.600 g, 4.190 mmol) and N,N-diisopropylethylamine (1.210 mL, 6.980 mmol) in DMF (20 mL) as described in the synthesis of step-7 of example-1 to afford the title compound (0.900 g, 75%) as a solid. LCMS: m/z 433.0 [M+2]+; ¹HNMR (400 MHz, Chloroform-d) δ 8.80-8.72 (d, J=1.7 Hz, 1H), 8.00-7.91 (dd, J=4.4, 1.7 Hz, 1H), 7.67-7.63 (d, J=1.4 Hz, 1H), 7.63-7.55 (m, 2H), 7.41-7.35 (dd, J=9.5, 1.8 Hz, 1H), 7.17-7.09 (m, 2H), 6.84-6.69 (m, 1H), 5.12-4.88 (tt, J=8.0, 8.0, 3.6, 3.6 Hz, 1H), 4.54-4.34 (t, J=4.8, 4.8 Hz, 2H), 4.28-4.11 (dd, J=10.3, 3.8 Hz, 1H), 4.14-3.96 (dd, J=10.3, 3.3 Hz, 1H), 3.31-3.13 (d, J=8.7 Hz, 2H).

### Step-5: N-((5-(((6′-fluoro-[2,3′-bipyridin]-3-yl)oxy)methyl)-4,5-dihydroisoxazol-3-yl)methyl)imidazo[1,2-a]pyridine-6-carboxamide (±)

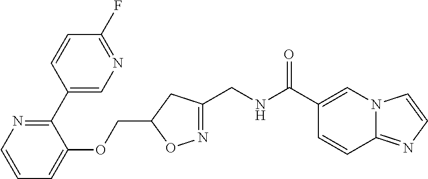 [SMILES: O=C(NCC1=NOC(COc2cccnc2-c2ccc(F)nc2)C1)c1ccc2nccn2c1]

N-((5-(((2-bromopyridin-3-yl)oxy)methyl)-4,5-dihydroisoxazol-3-yl)methyl)imidazo-[1,2-a]pyridine-6-carboxamide (±) (product of step-4, 0.450 g, 1.050 mmol) was reacted with (6-fluoropyridin-3-yl)boronic acid (0.222 g, 1.570 mmol) in presence of potassium carbonate (0.363 g, 1.100 mmol) and [1,1′-bis(diphenylphosphino)ferrocene]-dichloropalladium(II) complex with dichloromethane (0.086 g, 0.110 mmol) in 1,4-dioxane:water (9:1) (20 mL) for 16 h at 90° C. as described in the synthesis of step-8 of example-1. The crude product obtained was purified by preparative HPLC to get the title compound (0.092 g, 36% W/W) as a white solid. LCMS: m/z 447.1 [M+H]⁺; HPLC: 93.38%; ¹HNMR (400 MHz, Chloroform-d) δ 9.05-8.98 (d, J=1.7 Hz, 1H), 8.88-8.78 (d, J=2.5 Hz, 1H), 8.70 (s, 1H), 8.61-8.52 (ddd, J=8.6, 7.6, 2.5 Hz, 1H), 8.40-8.33 (dd, J=3.4, 2.5 Hz, 1H), 7.82-7.74 (dd, J=9.5, 1.9 Hz, 1H), 7.73-7.60 (m, 3H), 7.34-7.29 (m, 1H), 7.21-7.01 (dd, J=8.6, 2.5 Hz, 1H), 5.15-5.00 (dd, J=12.0, 6.7 Hz, 1H), 4.60-4.46 (dd, J=16.7, 5.9 Hz, 1H), 4.43-4.27 (m, 2H), 4.22-4.08 (dd, J=10.1, 1.8 Hz, 1H), 3.46-3.29 (dd, J=17.5, 12.1 Hz, 1H), 3.24-3.10 (dd, J=17.5, 6.7 Hz, 1H).

Preparative HPLC Method: Column: Zorbax Eclipse XDB C18 (21.2×250 mm 5 μm); 10 mm NH₄OAc in Water (A), Acetonitrile (B); Isocratic: 90:10.

Further the racemic mixture (product of step-5, 0.080 g) was separated by chiral preparative HPLC to afford two separated enantiomers (Examples 4a & 4b). Method: Column: Phenomenex Lux Cellulose-4 (250 mm×810 mm×5 μm); Hexane: 0.1% DEA in MeOH: Ethanol: ISOCRATIC (50:50); Flow Rate: 7 mL/min.

### Example-4a: N-((5-(((6′-fluoro-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.029 g; Chiral HPLC: 96.15% (Retention Time-10.948 min), HPLC: 94.07%; LCMS: m/z 447.1 [M+H]⁺.

### Example-4b: N-((5-(((6′-fluoro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.029 g; Chiral HPLC: 96.74% (Retention Time-14.71 min), HPLC: 97.04%; LCMS: m/z 447.1 [M+H]+.

### Example-5: N-((5-(((2-(1-methyl-1H-pyrazol-4-yl)pyridin-3-yl)oxy)methyl)-4,5-dihydroisoxazol-3-yl)methyl)imidazo[1,2-a]pyridine-6-carboxamide (±)

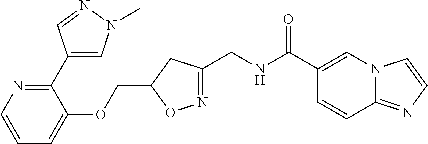 [SMILES: Cn1cc(-c2ncccc2OCC2CC(CNC(=O)c3ccc4nccn4c3)=NO2)cn1]

N-((5-(((2-bromopyridin-3-yl)oxy)methyl)-4,5-dihydroisoxazol-3-yl)methyl)imidazo-[1,2-a]-pyridine-6-carboxamide (±) (product of step-4 of example-4, 0.450 g, 1.050 mmol) was reacted with 1-methyl-4-(4,4,5,5-tetramethyl-1,3,2-dioxaborolan-2-yl)-1H-pyrazole (0.328 g, 1.570 mmol) in presence of potassium carbonate (0.363 g, 2.620 mmol) and [1,1′-bis(diphenylphosphino)ferrocene]dichloropalladium(II) complex with dichloromethane (0.086 g, 0.105 mmol) in 1,4-dioxane:water (9:1) (20 mL) as described in the synthesis of step-8 of example-1. The crude product obtained was purified by column chromatography on neutral alumina (dichloromethane/methanol=90/5) to afford the title compound (0.045 g, 10%) as an off white solid. LCMS: m/z 432.2 [M+H]; HPLC: 94.95%; ¹H NMR (400 MHz, Chloroform-d) δ 8.82-8.76 (d, J=1.4 Hz, 1H), 8.24-8.18 (dd, J=4.8, 1.3 Hz, 1H), 8.18 (s, 1H), 8.09 (s, 1H), 7.75-7.67 (d, J=1.3 Hz, 1H), 7.67-7.57 (m, 2H), 7.50-7.43 (dd, J=9.4, 1.9 Hz, 1H), 7.40 (s, 1H), 7.22-7.16 (dd, J=8.2, 1.3 Hz, 1H), 7.14-7.06 (dd, J=8.3, 4.7 Hz, 1H), 5.16-5.05 (t, J=9.4, 9.4 Hz, 1H), 4.54-4.29 (m, 3H), 4.15-4.02 (dd, J=10.3, 2.9 Hz, 1H), 3.96 (s, 3H), 3.40-3.23 (dd, J=17.4, 11.4 Hz, 1H), 3.23-3.06 (m, 1H).

### Example-6: N-((5-(((2-(3-fluoro-4-(methylcarbamoyl)phenyl)pyridin-3-yl)oxy)methyl)-4,5-dihydroisoxazol-3-yl)methyl)imidazo[1,2-a]pyridine-6-carboxamide (±)

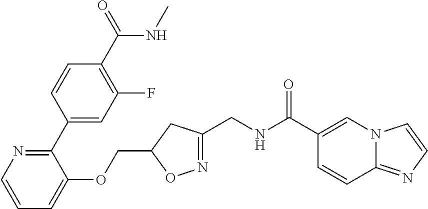 [SMILES: CNC(=O)c1ccc(-c2ncccc2OCC2CC(CNC(=O)c3ccc4nccn4c3)=NO2)cc1F]

N-((5-(((2-Bromopyridin-3-yl)oxy)methyl)-4,5-dihydroisoxazol-3-yl)methyl)imidazo-[1,2-a]pyridine-6-carboxamide (±) (product of step-4 of example-4, 0.200 g, 0.464 mmol) was reacted with 2-fluoro-N-methyl-4-(4,4,5,5-tetramethyl-1,3,2-dioxaborolan-2-yl)benzamide (0.155 g, 0.557 mmol) in presence of potassium carbonate (0.192 g, 1.392 mmol) and [1,1′-bis(diphenylphosphino)ferrocene]dichloropalladium(II) complex with dichloromethane (0.037 g, 0.046 mmol) in 1,4-dioxane:water (4:1) (12 mL) at 100° C. for 16 h as step-8 of example-1. The crude product obtained was purified by combiflash column chromatography (methanol/dichloromethane=9/91) to afford the title compound (0.061 g, 26%) as a solid. LCMS: m/z 503.2 [M+H]⁺; HPLC: 94.03%; ¹HNMR (400 MHz, DMSO-d₆) δ 9.13 (s, 1H), 8.99-8.93 (t, J=5.7, 5.7 Hz, 1H), 8.32-8.26 (d, J=4.4 Hz, 2H), 8.07 (s, 1H), 7.88-7.77 (m, 2H), 7.71-7.58 (m, 5H), 7.45-7.37 (dd, J=8.4, 4.6 Hz, 1H), 4.97-4.89 (dtd, J=11.1, 7.4, 7.2, 4.0 Hz, 1H), 4.27-4.11 (m, 4H), 3.25-3.13 (dd, J=17.6, 11.1 Hz, 1H), 2.93-2.84 (dd, J=17.5, 7.5 Hz, 1H), 2.82-2.74 (d, J=4.6 Hz, 3H).

### Example-7: N-((5-(([2,3′-bipyridin]-3-yloxy)methyl)-4,5-dihydroisoxazol-3-yl)methyl)-imidazo[1,2-a]pyridine-6-carboxamide (±)

### Step-1: Tert-butyl((5-(([2,3′-bipyridin]-3-yloxy)methyl)-4,5-dihydroisoxazol-3-yl)methyl)-carbamate (±)

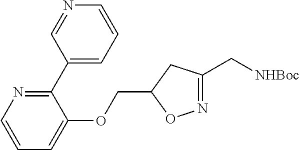 [SMILES: CC(C)(C)OC(=O)NCC1=NOC(COc2cccnc2-c2cccnc2)C1]

Tert-butyl((5-(((2-bromopyridin-3-yl)oxy)methyl)-4,5-dihydroisoxazol-3-yl)methyl)-carbamate (±) (product of step-2 of example-4, 0.500 g, 1.294 mmol) was reacted with pyridine-3-boronic acid (0.198 g, 1.553 mmol) in presence of potassium carbonate (0.535 g, 3.883 mmol) and [1,1′-bis(diphenylphosphino)ferrocene]dichloropalladium(II) complex with dichloromethane (0.105 g, 0.129 mmol) in 1,4-dioxane:water (4:1) (20 mL) at 100° C. for 16 h as described in the synthesis of step-4 of example-3. The crude product obtained was purified by combiflash column chromatography (hexanes/ethyl acetate=10/90) to get the title compound (0.390 g, 78%) as a solid which was further taken to the next step.

### Step-2: (5-(([2,3′-bipyridin]-3-yloxy)methyl)-4,5-dihydroisoxazol-3-yl)methanamine hydrochloride (±)

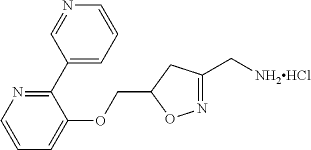

Tert-butyl((5-(([2,3′-bipyridin]-3-yloxy)methyl)-4,5-dihydroisoxazol-3-yl)methyl)-carbamate (±) (product of step-1, 0.390 g, 1.015 mmol) was reacted with dioxane.HCl as described in step-5 of example-3 to afford the title compound (0.320 g, 98.5%).

### Step-3: N-((5-(([2,3′-bipyridin]-3-yloxy)methyl)-4,5-dihydroisoxazol-3-yl)methyl)imidazo-[1,2-a]pyridine-6-carboxamide (±)

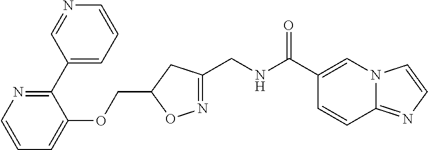 [SMILES: O=C(NCC1=NOC(COc2cccnc2-c2cccnc2)C1)c1ccc2nccn2c1]

(5-(([2,3′-Bipyridin]-3-yloxy)methyl)-4,5-dihydroisoxazol-3-yl)methanamine hydrochloride (±) (product of step-2, 0.320 g, 0.997 mmol) was reacted with imidazo[1,2-a]pyridine-6-carboxylic acid (0.193 g, 1.197 mmol) in presence of BOP reagent (0.485 g, 1.096 mmol) and DIPEA (0.525 mL, 2.991 mmol) in DMF (10 mL) at room temperature for 16 h. The reaction mixture was diluted with water (10 mL) extracted with ethyl acetate (50 mL) and washed with water (4×50 mL). The organic phase was dried over sodium sulphate and concentrated under reduced pressure to get a residue. The residue was purified by combiflash column chromatography (dichloromethane/methanol/triethylamine=91/8/1) to afford the title compound (0.150 g, 35%) as a solid. LCMS: m/z 428.7 [M+H]⁺; HPLC: 97.93%; ¹HNMR (400 MHz, DMSO-d₆): δ 9.16-9.12 (d, J=1.8 Hz, 1H), 9.11 (s, 1H), 9.05-8.99 (t, J=5.5, 5.5 Hz, 1H), 8.61-8.54 (dt, J=3.3, 1.5, 1.5 Hz, 1H), 8.37-8.31 (d, J=4.5 Hz, 1H), 8.30-8.25 (m, 1H), 8.11 (s, 1H), 7.68-7.60 (m, 4H), 7.51-7.45 (dd, J=7.9, 4.8 Hz, 1H), 7.45-7.39 (dd, J=8.4, 4.6 Hz, 1H), 5.01-4.91 (dq, J=10.4, 5.6, 5.6, 5.4 Hz, 1H), 4.28-4.12 (m, 4H), 3.27-3.15 (dd, J=17.6, 11.2 Hz, 1H), 3.00-2.86 (dd, J=17.7, 7.1 Hz, 1H).

Further the racemic mixture (product of step-3, 0.070 g) was separated by chiral preparative HPLC to get two separated enantiomers (Examples 7a & 7b). Method: Column: CHIRALPAK-IA (250×10) mm, 5 μm); Hexane: 0.1% DEA in Ethanol: ISOCRATIC (62:38); Flow Rate: 7 mL/min.

### Example-7a:N-((5-(([2,3′-bipyridin]-3-yloxy)methyl)-4,5-dihydroisoxazol-3-yl)methyl)-imidazo[1,2-a]pyridine-6-carboxamide (Isomer-1)

Yield: 0.018 g; Chiral HPLC: 98.54% (Retention Time-15.419 min), HPLC: 97.55%; LCMS: m/z 429.0 [M+H]⁺.

### Example-7b: N-((5-(([2,3′-bipyridin]-3-yloxy)methyl)-4,5-dihydroisoxazol-3-yl)methyl)-imidazo[1,2-a]pyridine-6-carboxamide ((Isomer-2)

Yield: 0.018 g; Chiral HPLC: 99.42% (Retention Time-17.955 min), HPLC: 99.42%; LCMS: m/z 429.0 [M+H]⁺.

### Example-8:N-((5-(((6′-chloro-[2,3′-bipyridin]-3-yl)oxy)methyl)-4,5-dihydroisoxazol-3-yl)methyl)imidazo[1,2-a]pyridine-6-carboxamide(±)

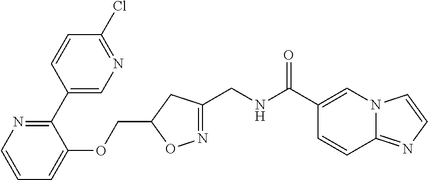 [SMILES: O=C(NCC1=NOC(COc2cccnc2-c2ccc(Cl)nc2)C1)c1ccc2nccn2c1]

N-((5-(((2-bromopyridin-3-yl)oxy)methyl)-4,5-dihydroisoxazol-3-yl)methyl)imidazo[1,2-a]pyridine-6-carboxamide (±) (product of step-4 of example-4, 0.100 g, 0.232 mmol) was reacted with 2-chloro-5-(4,4,5,5-tetramethyl-1,3,2-dioxaborolan-2-yl)pyridine (0.066 g, 0.278 mmol) in presence of potassium carbonate (0.096 g, 0.696 mmol) and [1,1′-bis(diphenylphosphino)ferrocene]dichloropalladium(II) complex with dichloromethane (0.019 g, 0.023 mmol) in 1,4-dioxane:water (4:1) (5 mL) for 16 h at 90° C. as described in the synthesis of step-8 of example-1. The crude product obtained was purified by preparative TLC (dichloromethane/methanol=95/5) to get the title compound (0.033 g, 30%) as a white solid. LCMS: m/z 463.3 [M+H]⁺; ¹H NMR (400 MHz, DMSO-d₆) δ 9.13 (d, J=1.7 Hz, 1H), 8.98 (t, J=5.8 Hz, 1H), 8.94 (d, J=2.4 Hz, 1H), 8.37-8.322 (m, 2H), 8.08 (d, J=1.2 Hz, 1H), 7.67-7.60 (m, 5H), 7.448 (dd, J=8.7, 2.9 Hz, 1H), 4.94 (d, J=13.6 Hz, 1H), 4.37-4.12 (m, 4H), 3.21 (dd, J=17.7, 11.2 Hz, 1H), 2.94 (dd, J=17.6, 7.3 Hz, 1H).

Further the racemic mixture (example-8, 0.024 g) was separated by chiral preparative HPLC to get two separated enantiomers (Examples 8a & 8b). Method: Column: Lux cellulose-4 (250×10) mm, 5 m); Hexane: [Ethanol: Isopropyl alcohol(8:2)] ISOCRATIC (30:70); Flow Rate: 20 mL/min.

### Example-8a: N-((5-(((6′-chloro-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.008 g; Chiral HPLC: 98.71% (Retention Time-15.777 min), HPLC: 98.51%; LCMS: m/z 463.0 [M+H]⁺.

### Example-8b: N-((5-(((6′-chloro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.008 g; Chiral HPLC: 92.67% (Retention Time-22.224 min), HPLC: 93.60%; LCMS: m/z 463.1 [M+H]⁺.

### Example-9: N-((5-(((5,6′-difluoro-[2,3′-bipyridin]-3-yl)oxy)methyl)-4,5-dihydroisoxazol-3-yl)methyl)imidazo[1,2-a]pyridine-6-carboxamide(±)

### Step-1: 3-(allyloxy)-2-chloro-5-fluoropyridine

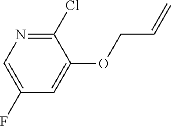

2-chloro-5-fluoropyridin-3-ol (0.500 g, 3.389 mmol) was reacted with allyl bromide in presence of potassium carbonate and potassium iodide in acetone as described in step-1 of example-3 to get the title compound (0.630 g, 99%) as liquid. LCMS: m/z 188.1 [M+1]+.

### Step-2: tert-butyl ((5-(((2-chloro-5-fluoropyridin-3-yl)oxy)methyl)-4,5-dihydroisoxazol-3-yl)methyl)carbamate(±)

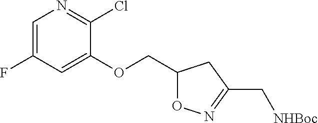 [SMILES: CC(C)(C)OC(=O)NCC1=NOC(COc2cc(F)cnc2Cl)C1]

3-(allyloxy)-2-chloro-5-fluoropyridine (product of step-1, 0.630 g, 3.368 mmol) was reacted with NCS in DMF as described in step-2 of example-4 to get the title compound (0.88 g, 66%) as a liquid. LCMS: m/z 360.2 [M+H]⁺.

### Step-3: tert-butyl ((5-(((5,6′-difluoro-[2,3′-bipyridin]-3-yl)oxy)methyl)-4,5-dihydroisoxazol-3-yl)methyl)carbamate(±)

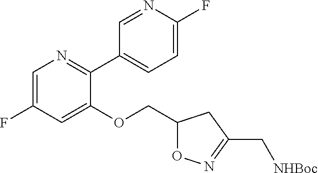 [SMILES: CC(C)(C)OC(=O)NCC1=NOC(COc2cc(F)cnc2-c2ccc(F)nc2)C1]

To a pre-purged (nitrogen, 15 min.) solution of tert-butyl((5-(((2-chloro-5-fluoropyridin-3-yl)oxy)methyl)-4,5-dihydroisoxazol-3-yl)methyl)carbamate(±) (product of step-2, 0.100 g, 0.278 mmol), (6-fluoropyridin-3-yl)boronic acid (0.047 g, 0.334 mmol) and K₃PO₄(0.176 g, 0.834 mmol) in 1,4-dioxane:H₂O (4:1) (5 ML) was added Pd₂(dba)₃ (0.0127 g, 0.0139 mmol) and PCy₃(0.0093 g, 0.033 mmol). Then the resulting reaction mixture was stirred at 100° C. for 16 h in sealed tube. After completion of the reaction by TLC the reaction mixture was filtered through celite pad. Filtrate was diluted with ethyl acetate (10 mL) and washed with brine, dried over Na₂SO₄, concentrated. The crude obtained was purified by column chromatography on silica gel (hexanes/ethyl acetate=40/40) to afford the title compound (0.050 g, 42.0%) as an off white solid. LCMS: m/z 421.3[M+H]⁺.

### Step-4: (5-(((5,6′-difluoro-[2,3′-bipyridin]-3-yl)oxy)methyl)-4,5-dihydroisoxazol-3-yl)methanamine hydrochloride (±)

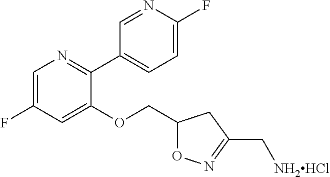

Tert-butyl ((5-(((5,6′-difluoro-[2,3′-bipyridin]-3-yl)oxy)methyl)-4,5-dihydroisoxazol-3-yl)methyl)carbamate(±) (product of step-3, 0.050 g, 0.118 mmol) was treated with 1,4-dioxane.HCl as described in step-5 of example-3. The solid obtained was washed with dry diethyl ether to afford the title compound (0.030 g, crude) as a solid. The crude product was taken to the next step without further purification.

### Step-5: N-((5-(((5,6′-difluoro-[2,3′-bipyridin]-3-yl)oxy)methyl)-4,5-dihydroisoxazol-3-yl)methyl)imidazo[1,2-a]pyridine-6-carboxamide (±)

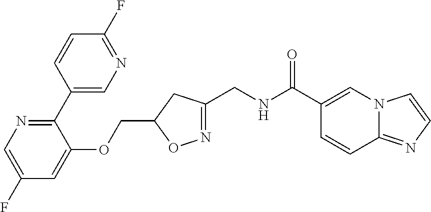 [SMILES: O=C(NCC1=NOC(COc2cc(F)cnc2-c2ccc(F)nc2)C1)c1ccc2nccn2c1]

(5-(((5,6′-Difluoro-[2,3′-bipyridin]-3-yl)oxy)methyl)-4,5-dihydroisoxazol-3-yl)-methanaminehydrochloride (±) (product of step-4, 0.030 g, 0.084 mmol) was reacted with imidazo[1,2-a]pyridine-6-carboxylic acid in presence of BOP reagent and DIPEA in DMF as described in step-3 of example-7. The crude obtained was purified by preparative HPLC [Method: Column: X bridge C-18(19 mm×150 mm), Water: Acetonitrile(1:1), Flow rate: 5 ml/min] to afford the title compound (0.015 g, 38%). LCMS: m/z 465.3 [M+H]⁺. ¹H NMR (400 MHz, DMSO-d6) δ 9.13 (d, J=1.7 Hz, 1H), 8.97 (t, J=5.8 Hz, 1H), 8.71 (d, J=2.4 Hz, 1H), 8.42 (td, J=8.4, 2.6 Hz, 1H), 8.34 (d, J=2.2 Hz, 1H), 8.08 (d, J=1.2 Hz, 1H), 7.73 (dd, J=10.8, 2.3 Hz, 1H), 7.69-7.59 (m, 3H), 7.28 (dd, J=8.7, 2.9 Hz, 1H), 4.94 (d, J=13.6 Hz, 1H), 4.37-4.12 (m, 4H), 3.22 (dd, J=17.7, 11.2 Hz, 1H), 2.89 (dd, J=17.6, 7.3 Hz, 1H).

Further the racemic mixture (product of step-5, 0.170 g) was separated by chiral preparative HPLC to get two separated enantiomers (Examples 9a & 9b). Method: Column: Lux cellulose-4 (250×10) mm, 5 m); Hexane: [Ethanol:Isopropyl alcohol(8:2)] ISOCRATIC (50:50); Flow Rate: 20 mL/min.

### Example-9a: N-((5-(((5,6′-difluoro-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.050 g; Chiral HPLC: 96.47% (Retention Time-11.691 min), HPLC: 96.11; LCMS: m/z 465.3 [M+H]+.

### Example-9b: N-((5-(((5,6′-difluoro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.070 g; Chiral HPLC:97.42% (Retention Time-15.442 min), HPLC: 97.71; LCMS: m/z 465.3 [M+H]+.

### Example-10: N-((5-(((6-cyano-6′-fluoro-[2,3′-bipyridin]-3-yl)oxy)methyl)-4,5-dihydroisoxazol-3-yl)methyl)imidazo[1,2-a]pyridine-6-carboxamide(±)

### Step-1: 3-(allyloxy)-2-chloro-6-iodopyridine

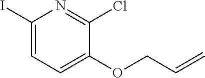

2-Chloro-6-iodopyridin-3-ol (4.65 g, 18.199 mmol) was reacted with allyl bromide in presence of potassium carbonate and potassium iodide in acetone as described in step-1 of example-3 to get the title compound (5.3 g, 98%) as a liquid. The product was taken to the next step without further purification.

### Step-2: tert-butyl ((5-(((2-chloro-6-iodopyridin-3-yl)oxy)methyl)-4,5-dihydroisoxazol-3-yl)methyl)carbamate (±)

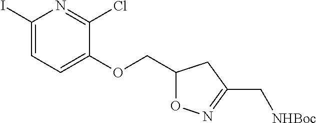 [SMILES: CC(C)(C)OC(=O)NCC1=NOC(COc2ccc(I)nc2Cl)C1]

3-(Allyloxy)-2-chloro-6-iodopyridine (product of step-1, 5.3 g, 17.935 mmol) was reacted with NCS in DMF as described in step-2 of example-4 to get the title compound (5.1 g, 61%) as a liquid. LCMS: m/z 368.1 [M-100]⁺.

### Step-3: tert-butyl ((5-(((2-chloro-6-cyanopyridin-3-yl)oxy)methyl)-4,5-dihydroisoxazol-3-yl)methyl)carbamate (±)

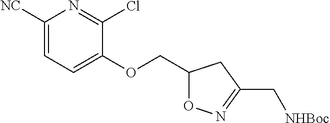 [SMILES: CC(C)(C)OC(=O)NCC1=NOC(COc2ccc(C#N)nc2Cl)C1]

To a solution of tert-butyl ((5-(((2-chloro-6-iodopyridin-3-yl)oxy)methyl)-4,5-dihydroisoxazol-3-yl)methyl)carbamate (±) (product of step-2, 0.500 g, 1.069 mmol) and zinc cyanide (0.094 g, 0.801 mmol) in DMF was purged argon for 15 min. followed by addition of Tetrakis(0.098 g, 0.085 mmol) in reaction tube, sealed and stirred at 80° C. for 2 h. After completion of the reaction by TLC, the reaction mixture was filtered through celite pad. Filtrate was dried over Na₂SO₄, concentrated. The crude obtained was purified by column chromatography on silica gel (hexanes/ethyl acetate=60/40) to get the title compound (0.240 g, crude) as an off white solid. LCMS: m/z 267.2 [M-100]⁺.

### Step-4: tert-butyl ((5-(((6-cyano-6′-fluoro-[2,3′-bipyridin]-3-yl)oxy)methyl)-4,5-dihydroisoxazol-3-yl)methyl)carbamate (±)

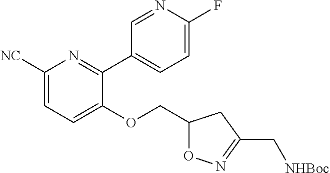 [SMILES: CC(C)(C)OC(=O)NCC1=NOC(COc2ccc(C#N)nc2-c2ccc(F)nc2)C1]

Tert-butyl((5-(((2-chloro-6-cyanopyridin-3-yl)oxy)methyl)-4,5-dihydroisoxazol-3-yl)methyl)carbamate (±) (product of step-3, 0.240 g, 0.653 mmol) was treated with (6-fluoropyridin-3-yl)boronic acid in presence of Pd₂(dba)₃, PCy₃ and K₃PO₄ in 1,4-dioxane:H₂O (4:1) as described in step-3 of example-9. The crude obtained was purified by column chromatography on silica gel (hexanes/ethyl acetate=20/80) to get the title compound (0.090 g, crude) as an off white solid. LCMS: m/z 328.3[M-100]+.

### Step-5: 3-((3-(aminomethyl)-4,5-dihydroisoxazol-5-yl)methoxy)-6′-fluoro-[2,3′-bipyridine]-6-carbonitrile hydrochloride(±)

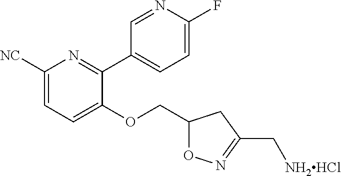

Tert-butyl((5-(((6-cyano-6′-fluoro-[2,3′-bipyridin]-3-yl)oxy)methyl)-4,5-dihydro-isoxazol-3-yl)methyl)carbamate(±) (product of step-4, 0.090 g, crude) was treated with 1,4-dioxane.HCl as described in step-5 of example-3 to get the title compound (0.060 g, crude) as a solid. The crude product was taken to the next step without further purification.

### Step-6: N-((5-(((6-cyano-6′-fluoro-[2,3′-bipyridin]-3-yl)oxy)methyl)-4,5-dihydroisoxazol-3-yl)methyl)imidazo[1,2-a]pyridine-6-carboxamide(±)

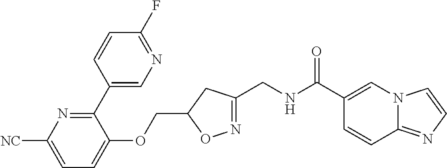 [SMILES: N#Cc1ccc(OCC2CC(CNC(=O)c3ccc4nccn4c3)=NO2)c(-c2ccc(F)nc2)n1]

3-((3-(Aminomethyl)-4,5-dihydroisoxazol-5-yl)methoxy)-6′-fluoro-[2,3′-bipyridine]-6-carbonitrile hydrochloride(±) (product of step-5, 0.060 g, 0.164 mmol) was reacted with imidazo[1,2-a]pyridine-6-carboxylic acid in presence of BOP reagent and DIPEA in DMF as described in step-3 of example-7. The crude obtained was purified by preparative TLC(dichloromethane/methanol=95/5×3) to get the title compound (0.020 g, 26%). LCMS: m/z 472.0 [M+H]⁺. ¹H NMR (400 MHz, DMSO-d6) δ 9.12 (t, J=1.4 Hz, 1H), 8.95 (t, J=5.7 Hz, 1H), 8.74 (d, J=2.4 Hz, 1H), 8.45 (td, J=8.3, 2.5 Hz, 1H), 8.15-8.02 (m, 2H), 7.81 (d, J=8.7 Hz, 1H), 7.64 (dd, J=14.2, 1.3 Hz, 3H), 7.32 (dd, J=8.6, 2.8 Hz, 1H), 4.95 (d, J=10.5 Hz, 1H), 4.45-4.05 (m, 4H), 3.28-3.09 (m, 1H), 2.90 (dd, J=17.7, 7.3 Hz, 1H).

Further the racemic mixture (product of step-6, 0.600 g) was separated by chiral preparative HPLC to get two separated enantiomers (Examples 10a & 10b). Method: Column: Lux cellulose-4 (250×10) mm, 5 μm); Hexane: [Methanol: Isopropyl alcohol(8:2)] ISOCRATIC (70:30); Flow Rate: 10 mL/min.

Example-10a: N-((5-(((6-cyano-6′-fluoro-[2,3′-bipyridin]-3-yl)oxy)methyl)-4,5-dihydroisoxazol-3-yl)methyl)imidazo[1,2-a]pyridine-6-carboxamide (Isomer-1). Yield: 0.270 g; Chiral HPLC 98.70% (Retention Time-16.431 min), HPLC: 95.78; LCMS: m/z 472.3 [M+H]⁺.

### Example-10b: N-((5-(((6-cyano-6′-fluoro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.180 g; Chiral HPLC: 95.83% (Retention Time-18.691 min), HPLC: 91.63; LCMS: m/z 472.3 [M+H]⁺.

### Example-11: N-((5-(((6′-fluoro-5-methyl-[2,3′-bipyridin]-3-yl)oxy)methyl)-4,5-dihydroisoxazol-3-yl)methyl)imidazo[1,2-a]pyridine-6-carboxamide (±)

### Step-1: 3-(allyloxy)-2-chloro-5-methylpyridine

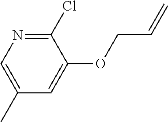

2-Chloro-5-methylpyridin-3-ol (1.5 g, 10.447 mmol) was reacted with allyl bromide in presence of potassium carbonate and potassium iodide in acetone as described in step-1 of example-3. The crude product obtained was purified by combiflash column chromatography (hexanes/ethyl acetate=75/25) to get the title compound (1.56 g, 80%). LCMS: m/z 184.2 [M+H]⁺.

### Step-2: tert-butyl ((5-(((2-chloro-5-methylpyridin-3-yl)oxy)methyl)-4,5-dihydroisoxazol-3-yl)methyl)carbamate(±)

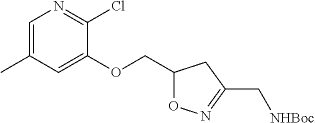 [SMILES: Cc1cnc(Cl)c(OCC2CC(CNC(=O)OC(C)(C)C)=NO2)c1]

3-(Allyloxy)-2-chloro-5-methylpyridine (product of step-1, 1.55 g, 8.44 mmol) was reacted with NCS in DMF as described in the synthesis of step-2 of example-4 to afford the title compound (1.45 g, 48%) as an off white solid. The product was taken to the next step without any analysis.

### Step-3: tert-butyl ((5-(((6′-fluoro-5-methyl-[2,3′-bipyridin]-3-yl)oxy)methyl)-4,5-dihydroisoxazol-3-yl)methyl)carbamate(±)

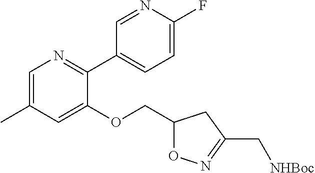 [SMILES: Cc1cnc(-c2ccc(F)nc2)c(OCC2CC(CNC(=O)OC(C)(C)C)=NO2)c1]

Tert-butyl((5-(((2-chloro-5-methylpyridin-3-yl)oxy)methyl)-4,5-dihydroisoxazol-3-yl)methyl)carbamate (±) (product of step-2, 0.500 g, 1.405 mmol) was treated with (6-fluoropyridin-3-yl)boronic acid in presence of Pd₂(dba)₃, PCy₃ and K₃PO₄ in 1,4-dioxane:H₂O (4:1) as described in step-3 of example-9 to get the title compound (0.350 g, 60%) as an off white solid. LCMS: m/z 417.1 [M+H]⁺.

### Step-4: (5-(((6′-fluoro-5-methyl-[2,3′-bipyridin]-3-yl)oxy)methyl)-4,5-dihydroisoxazol-3-yl)methanamine hydrochloride (±)

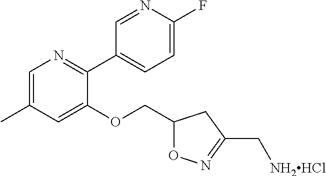

Tert-butyl((5-(((6′-fluoro-5-methyl-[2,3′-bipyridin]-3-yl)oxy)methyl)-4,5-dihydro-isoxazol-3-yl)methyl)carbamate (±) (product of step-3, 0.350 g, 0.840 mmol) was treated with 1,4-dioxane.HCl as described in the synthesis of step-5 of example-3 to get the title compound (0.250 g, crude) as a solid. The crude product was taken to the next step without further purification.

### Step-5: N-((5-(((6′-fluoro-5-methyl-[2,3′-bipyridin]-3-yl)oxy)methyl)-4,5-dihydroisoxazol-3-yl)methyl)imidazo[1,2-a]pyridine-6-carboxamide (±)

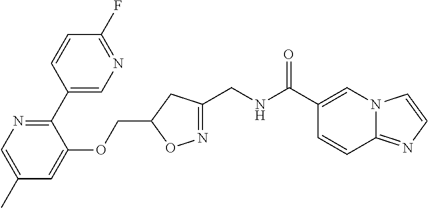 [SMILES: Cc1cnc(-c2ccc(F)nc2)c(OCC2CC(CNC(=O)c3ccc4nccn4c3)=NO2)c1]

(5-(((6′-Fluoro-5-methyl-[2,3′-bipyridin]-3-yl)oxy)methyl)-4,5-dihydroisoxazol-3-yl)methanamine hydrochloride (±) (product of step-4, 0.250 g, 0.708 mmol) was reacted with imidazo[1,2-a]pyridine-6-carboxylic acid in presence of BOP reagent and DIPEA in DMF as described in step-3 of example-7 to get the title compound (0.120 g, 37%). LCMS: m/z 461.3 [M+H]⁺. ¹H NMR (400 MHz, DMSO-d6) δ 9.13 (t, J=1.4 Hz, 1H), 8.97 (t, J=5.7 Hz, 1H), 8.75 (d, J=2.4 Hz, 1H), 8.46 (td, J=8.4, 2.5 Hz, 1H), 8.19-8.13 (m, 1H), 8.07 (d, J=1.3 Hz, 1H), 7.70-7.58 (m, 3H), 7.53-7.46 (m, 1H), 7.25 (dd, J=8.7, 2.8 Hz, 1H), 4.95 (td, J=10.5, 5.0 Hz, 1H), 4.34-4.06 (m, 4H), 3.21 (dd, J=17.6, 11.1 Hz, 1H), 2.89 (dd, J=17.6, 7.3 Hz, 1H), 2.33 (s, 3H).

Further the racemic mixture (product of step-5, 0.090 g) was separated by chiral preparative HPLC to get two separated enantiomers (Examples 11a & 11b). Method: Column: Lux cellulose-4 (250×10) mm, 5 μm); Hexane: Ethanol; ISOCRATIC (40:60); Flow Rate: 9 mL/min.

### Example-11a: N-((5-(((6′-fluoro-5-methyl-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.045 g; Chiral HPLC: 96.02% (Retention Time-15.872 min), HPLC: 97.47; LCMS: m/z 460.9 [M+H]⁺.

### Example-11b: N-((5-(((6′-fluoro-5-methyl-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.035 g; Chiral HPLC 95.19% (Retention Time-19.250 min), HPLC: 95.74; LCMS: m/z 461.0[M+H]⁺.

### Example-12: N-((5-(((6′-fluoro-6-methyl-[2,3′-bipyridin]-3-yl)oxy)methyl)-4,5-dihydroisoxazol-3-yl)methyl)imidazo[1,2-a]pyridine-6-carboxamide (±)

### Step-1: 3-(allyloxy)-2-bromo-6-methylpyridine

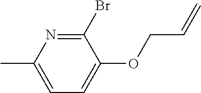

2-Bromo-6-methylpyridin-3-ol (2.0 g, 10.63 mmol) was reacted with allyl bromide in presence of potassium carbonate and potassium iodide in acetone as described in step-1 of example-3 to get the title compound (2.47 g, crude). The crude product was taken to the next step without further purification.

### Step-2: tert-butyl ((5-(((2-bromo-6-methylpyridin-3-yl)oxy)methyl)-4,5-dihydroisoxazol-3-yl)methyl)carbamate(±)

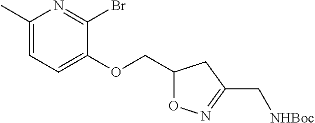 [SMILES: Cc1ccc(OCC2CC(CNC(=O)OC(C)(C)C)=NO2)c(Br)n1]

3-(Allyloxy)-2-bromo-6-methylpyridine (product of step-1, 2.47 g, 10.82 mmol) was reacted with NCS in DMF as described in step-2 of example-4 to get the title compound (1.4 g, 32%) as an off white solid. LCMS: m/z 402.2[M+H]⁺.

### Step-3: tert-butyl ((5-(((6′-fluoro-6-methyl-[2,3′-bipyridin]-3-yl)oxy)methyl)-4,5-dihydroisoxazol-3-yl)methyl)carbamate (±)

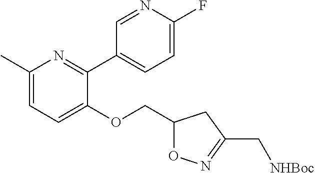 [SMILES: Cc1ccc(OCC2CC(CNC(=O)OC(C)(C)C)=NO2)c(-c2ccc(F)nc2)n1]

Tert-butyl ((5-(((2-bromo-6-methylpyridin-3-yl)oxy)methyl)-4,5-dihydroisoxazol-3-yl)methyl)carbamate (±) (product of step-2, 0.500 g, 1.249 mmol) was reacted with pyridin-3-ylboronic acid in presence of potassium carbonate and [1,1′-Bis(diphenylphosphino)-ferrocene]dichloropalladium(II) complex with dichloromethane as described in step-4 of example-3 to afford the titled compound (0.450 g, 86.0%) as an off white solid. LCMS: m/z 417.3 [M+H]⁺.

### Step-4: (5-(((6′-fluoro-6-methyl-[2,3′-bipyridin]-3-yl)oxy)methyl)-4,5-dihydroisoxazol-3-yl)methanamine hydrochloride (±)

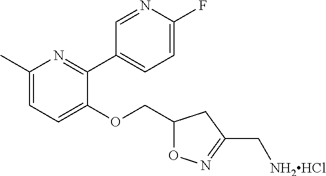

Tert-butyl ((5-(((6′-fluoro-6-methyl-[2,3′-bipyridin]-3-yl)oxy)methyl)-4,5-dihydroisoxazol-3-yl)methyl)carbamate (±) (product of step-3, 0.450 g, 1.08 mmol) was treated with 1,4-dioxane.HCl as described in step-5 of example-3 to afford the title compound (0.380 g, crude) as a solid. The crude product was taken to the next step without further purification.

### Step-5: N-((5-(((6′-fluoro-6-methyl-[2,3′-bipyridin]-3-yl)oxy)methyl)-4,5-dihydroisoxazol-3-yl)methyl)imidazo[1,2-a]pyridine-6-carboxamide(±)

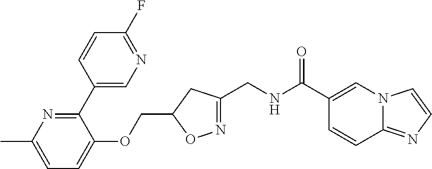 [SMILES: Cc1ccc(OCC2CC(CNC(=O)c3ccc4nccn4c3)=NO2)c(-c2ccc(F)nc2)n1]

(5-(((6′-Fluoro-6-methyl-[2,3′-bipyridin]-3-yl)oxy)methyl)-4,5-dihydroisoxazol-3-yl)methanaminehydrochloride (±) (product of step-4, 0.380 g, 1.077 mmol) was reacted with imidazo[1,2-a]pyridine-6-carboxylic acid in presence of BOP reagent and DIPEA in DMF as described in step-3 of example-7. The crude obtained was purified by preparative HPLC Method: Column Gemini NXC-18(21.2×250) mm, 10 mM Ammonium acetate in water:[Acetonitrile: Methanol(1:1)], Flow rate:15 ml/min to afford the title compound (0.120 g, 24%) as a white solid. LCMS: m/z 461.3 [M+H]⁺. ¹H NMR (400 MHz, DMSO-d6) δ 9.12 (t, J=1.4 Hz, 1H), 8.95 (q, J=7.0, 6.3 Hz, 1H), 8.75 (d, J=2.4 Hz, 1H), 8.46 (td, J=8.4, 2.5 Hz, 1H), 8.07 (d, J=1.3 Hz, 1H), 7.69-7.58 (m, 3H), 7.54 (d, J=8.6 Hz, 1H), 7.30-7.20 (m, 2H), 4.92 (ddq, J=10.7, 6.6, 4.0, 3.4 Hz, 1H), 4.30-4.00 (m, 4H), 3.26-3.10 (m, 1H), 2.88 (dd, J=17.6, 7.3 Hz, 1H), 2.46 (s, 3H).

Further the racemic mixture (product of step-5, 0.090 g) was separated by chiral preparative HPLC to afford two separated enantiomers (Examples 12a & 12b). Method: Column: Lux cellulose-4 (250×10) mm, 5 μm); Hexane: Ethanol; ISOCRATIC (30:70); Flow Rate: 9 mL/min.

### Example-12a: N-((5-(((6′-fluoro-6-methyl-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.040 g; Chiral HPLC: 98.5% (Retention Time-14.451 min), HPLC: 98.77%; LCMS: m/z 461.1 [M+H]⁺.

### Example-12b: N-((5-(((6′-fluoro-6-methyl-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.040 g; Chiral HPLC: 93.13% (Retention Time-20.649 min), HPLC: 99.57; LCMS: m/z 460.8 [M+H]⁺.

### Example-13: N-((5-(((5-chloro-[2,3′-bipyridin]-3-yl)oxy)methyl)-4,5-dihydroisoxazol-3-yl)methyl)imidazo[1,2-a]pyridine-6-carboxamide (±)

### Step-1: 3-(allyloxy)-5-chloro-2-iodopyridine

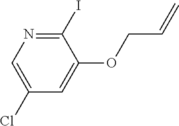

5-Chloro-2-iodopyridin-3-ol (synthesized as per US2012/238548 A1) (2.40 g, 9.391 mmol) reacted with allyl bromide in presence of potassium carbonate and potassium iodide in acetone as described in step-1 of example-3 to get the title compound (2.6 g, 93%) as a liquid. The product obtained was taken to the next step without further purification.

### Step-2: Tert-butyl ((5-(((5-chloro-2-iodopyridin-3-yl)oxy)methyl)-4,5-dihydroisoxazol-3-yl)methyl)carbamate (±)

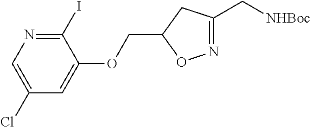 [SMILES: CC(C)(C)OC(=O)NCC1=NOC(COc2cc(Cl)cnc2I)C1]

3-(Allyloxy)-5-chloro-2-iodopyridine (product of step-1, 2.6 g, 8.798 mmol) was reacted with NCS in DMF as described in step-2 of example-4 to get the title compound (1.6 g, 39%) as a solid. LCMS: m/z 368.1[M+H]+.

### Step-3: Tert-butyl ((5-(((5-chloro-[2,3′-bipyridin]-3-yl)oxy)methyl)-4,5-dihydroisoxazol-3-yl)methyl)carbamate (±)

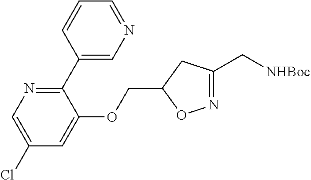 [SMILES: CC(C)(C)OC(=O)NCC1=NOC(COc2cc(Cl)cnc2-c2cccnc2)C1]

Tert-butyl((5-(((5-chloro-2-iodopyridin-3-yl)oxy)methyl)-4,5-dihydroisoxazol-3-yl)methyl)carbamate (±) (product of step-2, 0.400 g, 0.855 mmol) was reacted with pyridin-3-ylboronic acid in presence of potassium carbonate and [1,1′-Bis(diphenylphosphino)ferrocene]-dichloropalladium(II) complex with dichloromethane as described in the synthesis of step-4 of example-3 to give the titled compound (0.350 g, 97.0%) as an off white solid. LCMS: m/z 419.1[M+H]⁺.

### Step-4: (5-(((5-Chloro-[2,3′-bipyridin]-3-yl)oxy)methyl)-4,5-dihydroisoxazol-3-yl)methanamine hydrochloride (±)

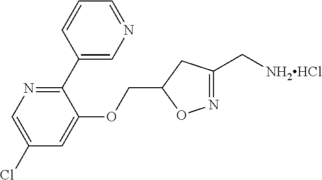

Tert-butyl((5-(((5-chloro-[2,3′-bipyridin]-3-yl)oxy)methyl)-4,5-dihydroisoxazol-3-yl)methyl)carbamate (±) (product of step-3, 0.350 g, 0.835 mmol) was treated with 1,4-dioxane.HCl as described in step-5 of example-3. The solid obtained was washed with dry diethyl ether to get the title compound (0.300 g, crude) as a solid. The crude product was taken to the next step without further purification.

### Step-5: N-((5-(((5-chloro-[2,3′-bipyridin]-3-yl)oxy)methyl)-4,5-dihydroisoxazol-3-yl)methyl)imidazo[1,2-a]pyridine-6-carboxamide (±)

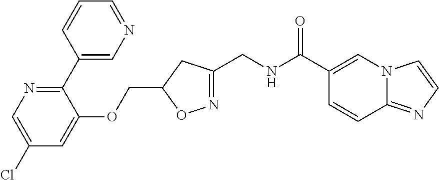 [SMILES: O=C(NCC1=NOC(COc2cc(Cl)cnc2-c2cccnc2)C1)c1ccc2nccn2c1]

(5-(((5-Chloro-[2,3′-bipyridin]-3-yl)oxy)methyl)-4,5-dihydroisoxazol-3-yl) methanamine hydrochloride (±) (product of step-4, 0.300 g, 0.838 mmol) was reacted with imidazo[1,2-a]pyridine-6-carboxylic acid in presence of BOP reagent and DIPEA in DMF as described in step-3 of example-7 to get the title compound (0.150 g, 37%) as a white solid. LCMS: m/z 463.3 [M+H]⁺. ¹H NMR (400 MHz, DMSO-d6) δ 9.14 (s, 1H), 9.09-8.97 (m, 2H), 8.58 (dd, J=4.6, 1.8 Hz, 1H), 8.37 (d, J=2.0 Hz, 1H), 8.25 (dt, J=8.0, 1.9 Hz, 1H), 8.08 (s, 1H), 7.84 (d, J=2.2 Hz, 1H), 7.71-7.60 (m, 3H), 7.49 (dd, J=8.0, 4.7 Hz, 1H), 4.96 (d, J=9.6 Hz, 1H), 4.41-4.11 (m, 4H), 3.21 (dd, J=17.7, 11.3 Hz, 1H), 2.90 (dd, J=17.4, 7.3 Hz, 1H).

Further the racemic mixture (product of step-5, 0.150 g) was separated by chiral preparative HPLC to get two separated enantiomers (Examples 13a & 13b). Method: Column: Lux cellulose-4 (250×10) mm, 5 m); Hexane: [Methanol: Isopropyl alcohol(1:1)] ISOCRATIC (50:50); Flow Rate: 9 mL/min.

### Example-13a: N-((5-(((5-chloro-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.045 g; Chiral HPLC: 98.35% (Retention Time-18.443 min), HPLC: 97.1%; LCMS: m/z 462.9 [M+H]⁺.

### Example-13b: N-((5-(((5-chloro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.055 g; Chiral HPLC: 98.5% (Retention Time-27.244 min), HPLC: 99.02%; LCMS: m/z 462.9 [M+H]⁺.

### Example-14: N-((5-(((5-chloro-6′-fluoro-[2,3′-bipyridin]-3-yl)oxy)methyl)-4,5-dihydro-isoxazol-3-yl)methyl)imidazo[1,2-a]pyridine-6-carboxamide (±)

### Step-1: tert-butyl((5-(((5-chloro-6′-fluoro-[2,3′-bipyridin]-3-yl)oxy)methyl)-4,5-dihydroisoxazol-3-yl)methyl)carbamate (±)

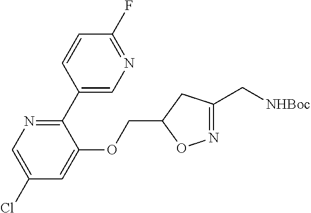 [SMILES: CC(C)(C)OC(=O)NCC1=NOC(COc2cc(Cl)cnc2-c2ccc(F)nc2)C1]

Tert-butyl((5-(((5-chloro-2-iodopyridin-3-yl)oxy)methyl)-4, 5-dihydroisoxazol-3-yl)-methyl) carbamate (±) (product of step-2 of example-13, 0.400 g, 0.855 mmol) was reacted with (6-fluoropyridin-3-yl)boronic acid in presence of potassium carbonate and [1,1′-Bis(diphenylphosphino)ferrocene]-dichloropalladium(II) complex with dichloromethane as described in step-4 of example-3 to afford the title compound (0.300 g, 80%) as an off white solid. LCMS: m/z 437.3[M+H]⁺.

### Step-2: (5-(((5-chloro-6′-fluoro-[2,3′-bipyridin]-3-yl)oxy)methyl)-4,5-dihydroisoxazol-3-yl)methanamine hydrochloride (±)

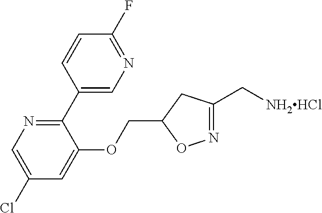

Tert-butyl((5-(((5-chloro-6′-fluoro-[2,3′-bipyridin]-3-yl)oxy)methyl)-4,5-dihydroisoxazol-3-yl)methyl)carbamate (±) (product of step-1,0.300 g, 0.686 mmol) was treated with 1,4-dioxane.HCl as described in step-5 of example-3. The solid obtained was washed with dry diethyl ether to afford the title compound (0.150 g, 58%) as a solid. The product obtained was taken to the next step.

### Step-3: N-((5-(((5-chloro-6′-fluoro-[2,3′-bipyridin]-3-yl)oxy)methyl)-4,5-dihydroisoxazol-3-yl)methyl)imidazo[1,2-a]pyridine-6-carboxamide (±)

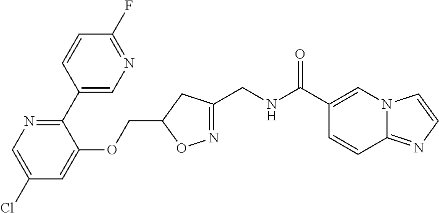 [SMILES: O=C(NCC1=NOC(COc2cc(Cl)cnc2-c2ccc(F)nc2)C1)c1ccc2nccn2c1]

(5-(((5-Chloro-6′-fluoro-[2,3′-bipyridin]-3-yl)oxy)methyl)-4,5-dihydroisoxazol-3-yl)methanamine hydrochloride (±) (product of step-2, 0.150 g, 0.401 mmol) was reacted with imidazo[1,2-a]pyridine-6-carboxylic acid in presence of BOP reagent and DIPEA in DMF as described in step-3 of example-7 to get the title compound (0.045 g, 23%) as a white solid. LCMS: m/z 481.3 [M+H]⁺. ¹H NMR (400 MHz, DMSO-d6) δ 9.13 (t, J=1.4 Hz, 1H), 8.97 (t, J=5.7 Hz, 1H), 8.75 (d, J=2.4 Hz, 1H), 8.46 (td, J=8.3, 2.5 Hz, 1H), 8.37 (d, J=2.0 Hz, 1H), 8.08 (d, J=1.3 Hz, 1H), 7.86 (d, J=2.0 Hz, 1H), 7.69-7.59 (m, 3H), 7.29 (dd, J=8.6, 2.8 Hz, 1H), 4.95 (ddt, J=10.7, 6.5, 3.7 Hz, 1H), 4.27 (ddd, J=38.1, 10.4, 4.4 Hz, 4H), 3.22 (dd, J=17.6, 11.1 Hz, 1H), 2.88 (dd, J=17.6, 7.2 Hz, 1H).

Further the racemic mixture (product of step-3, 0.420 g) was separated by chiral preparative HPLC to get two separated enantiomers (Examples 14a & 14b). Method: Column: Lux cellulose-4 (250×10) mm, 5 m); Hexane: 0.2% Diethyl amine in Ethanol: ISOCRATIC (50:50); Flow Rate: 8 mL/min.

### Example-14a: N-((5-(((5-chloro-6′-fluoro-[2,3′-bipyridin]-3-yl)oxy)methyl)-4,5-dihydroisoxazol-3-yl)methyl)imidazo[1,2-a]pyridine-6-carboxamide (Isomer-1)

Yield: 0.140 g; Chiral HPLC: 97.30% (Retention Time-15.257 min), HPLC: 96.26%; LCMS: m/z 481.2 [M+H]⁺.

### Example-14b: N-((5-(((5-chloro-6′-fluoro-[2,3′-bipyridin]-3-yl)oxy)methyl)-4,5-dihydroisoxazol-3-yl)methyl)imidazo[1,2-a]pyridine-6-carboxamide (Isomer-2)

Yield: 0.140 g; Chiral HPLC: 97.77% (Retention Time-18.959 min), HPLC: 97.90%; LCMS: m/z 480.9 [M+H]⁺.

### Example-15: N-((5-(((5-fluoro-[2,3′-bipyridin]-3-yl)oxy)methyl)-4,5-dihydroisoxazol-3-yl)methyl)imidazo[1,2-a]pyridine-6-carboxamide (±)

### Step-1: tert-butyl ((5-(((5-fluoro-[2,3′-bipyridin]-3-yl)oxy)methyl)-4,5-dihydroisoxazol-3-yl)methyl)carbamate (±)

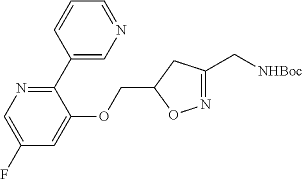 [SMILES: CC(C)(C)OC(=O)NCC1=NOC(COc2cc(F)cnc2-c2cccnc2)C1]

Tert-butyl ((5-(((2-chloro-5-fluoropyridin-3-yl)oxy)methyl)-4,5-dihydroisoxazol-3-yl)methyl)carbamate (±) (product of step-2 of example-9, 0.480 g, 1.337 mmol) was treated with pyridin-3-ylboronic acid in presence of Pd₂(dba)₃, PCy₃ and K₃PO₄ in 1,4-dioxane:H₂O (4:1) as described in step-3 of example-9. The crude obtained was purified by combiflash (hexanes/ethyl acetate=70/30) to afford the title compound (0.360 g, 66%) as an off white solid. The product obtained was taken to the next step.

### Step-2: (5-(((5-fluoro-[2,3′-bipyridin]-3-yl)oxy)methyl)-4,5-dihydroisoxazol-3-yl)methanamine hydrochloride (±)

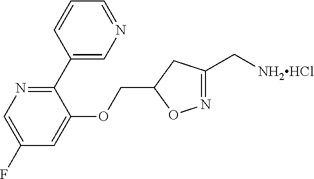

Tert-butyl ((5-(((5-fluoro-[2,3′-bipyridin]-3-yl)oxy)methyl)-4,5-dihydroisoxazol-3-yl)methyl)carbamate (±) (product of step-1, 0.360 g, 0.894 mmol) was treated with 1,4-dioxane.HCl as described in the synthesis of step-5 of example-3. The solid obtained was washed with dry diethyl to give title compound (0.300 g, crude) as a solid. The crude product was taken to the next step without further purification.

### Step-3: N-((5-(((5-fluoro-[2,3′-bipyridin]-3-yl)oxy)methyl)-4,5-dihydroisoxazol-3-yl)methyl)imidazo[1,2-a]pyridine-6-carboxamide (±)

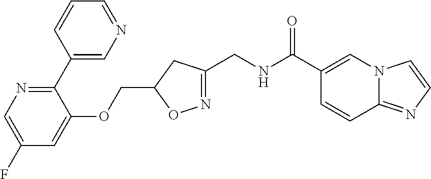 [SMILES: O=C(NCC1=NOC(COc2cc(F)cnc2-c2cccnc2)C1)c1ccc2nccn2c1]

(5-(((5-Fluoro-[2,3′-bipyridin]-3-yl)oxy)methyl)-4,5-dihydroisoxazol-3-yl) methanamine hydrochloride (±) (product of step-2, 0.300 g, 0.885 mmol) was reacted with imidazo[1,2-a]pyridine-6-carboxylic acid in presence of BOP reagent and DIPEA in DMF as described in step-3 of example-7 to afford the title compound (0.210 g, 53.2%). LCMS: m/z 446.9 [M+H]⁺. ¹H NMR (400 MHz, DMSO-d6) δ 9.14 (t, J=1.4 Hz, 1H), 9.07-8.98 (m, 2H), 8.57 (dd, J=4.8, 1.7 Hz, 1H), 8.38-8.31 (m, 1H), 8.22 (dt, J=8.1, 2.0 Hz, 1H), 8.12-8.05 (m, 1H), 7.72 (dd, J=10.9, 2.4 Hz, 1H), 7.68-7.59 (m, 3H), 7.49 (ddd, J=8.0, 4.8, 0.9 Hz, 1H), 5.06-4.86 (m, 1H), 4.36-4.11 (m, 4H), 3.22 (dd, J=17.6, 11.1 Hz, 1H), 2.90 (dd, J=17.6, 7.2 Hz, 1H).

### Example-16: 1-((5-(((6′-fluoro-[2,3′-bipyridin]-3-yl)oxy)methyl)-4,5-dihydroisoxazol-3-yl)methyl)-3-(2-methylpyridin-4-yl)urea (±)

### Step-1: Phenyl(2-methylpyridin-4-yl)carbamate

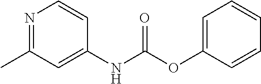 [SMILES: Cc1cc(NC(=O)Oc2ccccc2)ccn1]

To a solution of 2-Methylpyridin-4-amine (0.150 g, 1.388 mmol) in pyridine (5 mL) was added phenylchloroformate (0.261 g, 1.66 mmol) at 0° C. The reaction mixture was stirred for 1 h, and then reaction mixture was diluted with water (50 mL) and extracted with ethyl acetate (2×50 mL). The combined organic layer was dried over anhydrous sodium sulphate and concentrated under reduced pressure to get the title compound (0.210 g, crude) as a solid. The crude product was taken to the next step without further purification.

### Step-2: Tert-butyl ((5-(((6′-fluoro-[2,3′-bipyridin]-3-yl)oxy)methyl)-4,5-dihydroisoxazol-3-yl)methyl)carbamate (±)

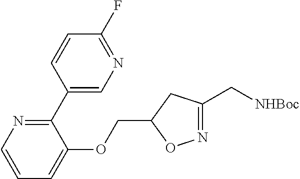 [SMILES: CC(C)(C)OC(=O)NCC1=NOC(COc2cccnc2-c2ccc(F)nc2)C1]

Tert-butyl((5-(((2-bromopyridin-3-yl)oxy)methyl)-4,5-dihydroisoxazol-3-yl)methyl)-carbamate (±) (product of step-2 of example-4, 5.00 g, 12.946 mmol) was reacted with (6-fluoropyridin-3-yl)boronic acid (2.180 g, 15.535 mmol) in presence of potassium carbonate (5.350 g, 38.838 mmol) and [1,1′-bis(diphenylphosphino)ferrocene]dichloropalladium(II) complex with dichloromethane (1.050 g, 1.294 mmol) in 1,4-dioxane:water (4:1) (62 mL) for 16 h at 100° C. as described in the synthesis of step-4 of example-3. The crude product obtained was purified by combiflash column chromatography (hexanes/ethyl acetate=30/70) to get the title compound (4.60 g, 88%) as a solid which was further taken to the next step without analysis.

### Step-3: (5-(((6′-fluoro-[2,3′-bipyridin]-3-yl)oxy)methyl)-4,5-dihydroisoxazol-3-yl)methanamine hydrochloride (±)

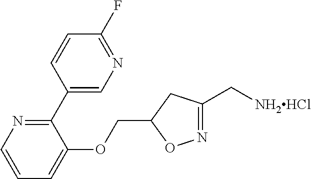

Tert-butyl((5-(((6′-fluoro-[2,3′-bipyridin]-3-yl)oxy)methyl)-4,5-dihydroisoxazol-3-yl)methyl) carbamate (±) (product of step-2, 4.60 g, 11.434 mmol) was reacted with dioxane.HCl as described in the synthesis of step-5 of example-3 to give title compound (3.800 g, 98.4%). LCMS: m/z 436.2 [M+H]⁺.

### Step-4: 1-((5-(((6′-fluoro-[2,3′-bipyridin]-3-yl)oxy)methyl)-4,5-dihydroisoxazol-3-yl)methyl)-3-(2-methylpyridin-4-yl)urea (±)

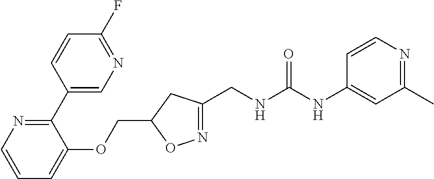 [SMILES: Cc1cc(NC(=O)NCC2=NOC(COc3cccnc3-c3ccc(F)nc3)C2)ccn1]

A solution of (5-(((6′-fluoro-[2,3′-bipyridin]-3-yl)oxy)methyl)-4,5-dihydroisoxazol-3-yl)methanamine hydrochloride (±) (product of step-3, 0.20 g, 0.590 mmol), phenyl(2-methylpyridin-4-yl)carbamate (product of step-1, 0.161 g, 0.708 mmol) and triethylamine (0.245 mL, 1.770 mmol) in DMSO (5 mL) was stirred at room temperature for 16 h under nitrogen atmosphere. The reaction mixture was diluted with water (20 mL), extracted with ethyl acetate (50 mL) and washed with water (4×50 mL). The organic phase was dried over sodium sulphate and concentrated under reduced pressure to give a residue. The residue was purified by combiflash column chromatography (dichloromethane/methanol=94/6) to give the title compound (0.061 g, 23%) as a solid. LCMS: m/z 436.7 [M+H]⁺; ¹HNMR (400 MHz, DMSO-d₆) δ 9.14 (s, 1H), 8.78-8.74 (d, J=2.4 Hz, 1H), 8.52-8.44 (td, J=8.4, 8.3, 2.5 Hz, 1H), 8.36-8.29 (dd, J=4.6, 1.2 Hz, 1H), 8.20-8.14 (d, J=5.6 Hz, 1H), 7.69-7.60 (dd, J=8.4, 1.3 Hz, 1H), 7.47-7.40 (dd, J=8.4, 4.6 Hz, 1H), 7.29-7.24 (m, 2H), 7.22-7.16 (dd, J=5.7, 2.1 Hz, 1H), 6.76-6.69 (t, J=5.7, 5.7 Hz, 1H), 5.00-4.89 (td, J=11.2, 11.1, 5.2 Hz, 1H), 4.27-4.13 (m, 2H), 4.06-3.95 (d, J=5.4 Hz, 2H), 3.23-3.12 (dd, J=17.5, 11.1 Hz, 1H), 2.98-2.86 (dd, J=17.5, 7.2 Hz, 1H), 2.41 (s, 3H).

### Example-17: 1-((5-(((6′-fluoro-[2,3′-bipyridin]-3-yl)oxy)methyl)-4,5-dihydroisoxazol-3-yl)methyl)-3-(pyridin-4-yl)urea (±)

### Step-1: Phenyl(pyridin-4-yl)carbamate

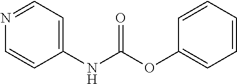 [SMILES: O=C(Nc1ccncc1)Oc1ccccc1]

4-Aminopyridine (0.300 g, 3.19 mmol) was reacted with phenylchloroformate (0.600 g, 3.83 mmol) in pyridine (5 mL) as described in step-1 of example-16 to get the title compound (0.5 g, crude) as a solid. The crude product was taken to the next step without further purification.

### Step-2: 1-((5-(((6′-fluoro-[2,3′-bipyridin]-3-yl)oxy)methyl)-4,5-dihydroisoxazol-3-yl)methyl)-3-(pyridin-4-yl)urea (±)

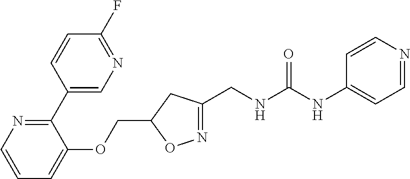 [SMILES: O=C(NCC1=NOC(COc2cccnc2-c2ccc(F)nc2)C1)Nc1ccncc1]

(5-(((6′-Fluoro-[2,3′-bipyridin]-3-yl)oxy)methyl)-4,5-dihydroisoxazol-3-yl) methanamine hydrochloride (±) (product of step-3 of example-16, 0.200 g, 0.590 mmol) was treated with phenyl(pyridin-4-yl)carbamate (product of step-1, 0.151 g, 0.708 mmol) in presence of triethylamine (0.245 mL, 1.770 mmol) in DMSO (5 mL) at room temperature for 16 h under nitrogen atmosphere as described in step-4 of example-16. The crude product obtained was purified by combiflash column chromatography (dichloromethane/methanol=94/6) to afford the title compound (0.028 g, 10%) as a solid. HPLC: 93.37%; LCMS: nm/z 422.7 [M+H]⁺; ¹H NMR (400 MHz, DMSO-d₆) δ 9.27 (s, 1H), 8.80-8.73 (d, J=2.5 Hz, 1H), 8.54-8.44 (td, J=8.4, 8.4, 2.5 Hz, 1H), 8.35-8.26 (ddd, J=8.2, 4.7, 1.5 Hz, 3H), 7.68-7.61 (dd, J=8.5, 1.4 Hz, 1H), 7.47-7.39 (dd, J=8.4, 4.6 Hz, 1H), 7.39-7.34 (m, 2H), 7.30-7.24 (dd, J=8.5, 2.8 Hz, 1H), 6.78-6.70 (t, J=5.7, 5.7 Hz, 1H), 5.00-4.88 (dd, J=11.3, 5.7 Hz, 1H), 4.29-3.96 (m, 4H), 3.24-3.11 (dd, J=17.6, 10.9 Hz, 1H), 2.97-2.84 (dd, J=17.5, 7.3 Hz, 1H).

### Example-18: 1-((5-(((6′-fluoro-[2,3′-bipyridin]-3-yl)oxy)methyl)-4,5-dihydroisoxazol-3-yl)methyl)-3-(pyridazin-4-yl)urea (i)

### Step-1: phenyl(pyridazin-4-yl)carbamate

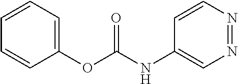 [SMILES: O=C(Nc1ccnnc1)Oc1ccccc1]

To a solution of pyridazin-4-amine hydrochloride (0.090 g, 0.680 mmol) and pyridine (0.120 g, 1.500 mmol) in acetonitrile:THF (1:1) (10 mL) was added phenylchloroformate (0.090 mL, 0.750 mmol) at 0° C. The above reaction mixture was stirred for 12 h at room temperature. The reaction mixture was diluted with water (50 mL) and extracted with ethyl acetate (2×50 mL). The combined organic layer was dried over sodium sulphate and concentrated under reduced pressure. The residue was purified by column chromatography (ethyl acetate/hexanes=60/40) to afford the title compound (0.100 g, 68%) as a solid.

### Step-2: 1-((5-(((6′-fluoro-[2,3′-bipyridin]-3-yl)oxy)methyl)-4,5-dihydroisoxazol-3-yl)methyl)-3-(pyridazin-4-yl)urea (±)

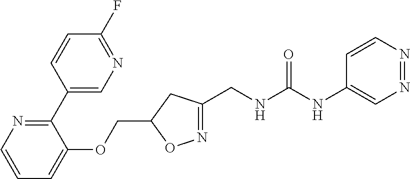 [SMILES: O=C(NCC1=NOC(COc2cccnc2-c2ccc(F)nc2)C1)Nc1ccnnc1]

(5-(((6′-Fluoro-[2,3′-bipyridin]-3-yl)oxy)methyl)-4,5-dihydroisoxazol-3-yl)methanamine hydrochloride (±) (product of step-3 of example-16, 0.200 g, 0.590 mmol) was treated with phenyl(pyridazin-4-yl)carbamate (product of step-1, 0.152 g, 0.708 mmol) in presence of triethylamine (0.245 mL, 1.770 mmol) in DMSO (5 mL) at room temperature for 16 h as described in step-4 of example-16. The crude product obtained was purified by combiflash column chromatography (dichloromethane/methanol=94/6) to afford the title compound (0.051 g, 20%) as a solid. LCMS: m/z 423.7 [M+H]⁺; ¹H NMR (400 MHz, DMSO-d₆) δ 9.49 (s, 1H), 9.15-9.11 (dd, J=2.6, 1.2 Hz, 1H), 8.91-8.85 (d, J=6.0 Hz, 1H), 8.79-8.74 (d, J=2.4 Hz, 1H), 8.53-8.44 (td, J=8.3, 8.3, 2.4 Hz, 1H), 8.37-8.29 (dd, J=4.5, 1.3 Hz, 1H), 7.77-7.72 (dd, J=6.0, 2.7 Hz, 1H), 7.67-7.61 (dd, J=8.4, 1.3 Hz, 1H), 7.47-7.40 (dd, J=8.4, 4.6 Hz, 1H), 7.29-7.23 (dd, J=8.6, 2.8 Hz, 1H), 7.03-6.94 (t, J=5.6, 5.6 Hz, 1H), 5.01-4.91 (dq, J=10.9, 5.2, 5.2, 4.3 Hz, 1H), 4.28-4.11 (m, 2H), 4.10-3.95 (d, J=5.5 Hz, 2H), 3.26-3.14 (dd, J=17.5, 11.1 Hz, 1H), 2.97-2.83 (dd, J=17.5, 7.2 Hz, 1H).

### Example-19: 1-((5-(((2-(1-methyl-1H-pyrazol-4-yl)pyridin-3-yl)oxy)methyl)-4,5-dihydroisoxazol-3-yl)methyl)-3-(2-methylpyridin-4-yl)urea (±)

### Step-1: Tert-butyl ((5-(((2-(1-methyl-1H-pyrazol-4-yl)pyridin-3-yl)oxy)methyl)-4,5-dihydroisoxazol-3-yl)methyl)carbamate (±)

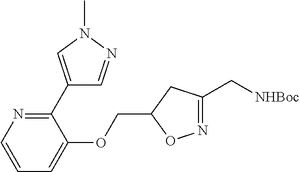 [SMILES: Cn1cc(-c2ncccc2OCC2CC(CNC(=O)OC(C)(C)C)=NO2)cn1]

Tert-butyl ((5-(((2-bromopyridin-3-yl)oxy)methyl)-4,5-dihydroisoxazol-3-yl)methyl)-carbamate (±) (product of step-2 of example-4, 3.00 g, 7.767 mmol) was reacted with 1-methyl-4-(4,4,5,5-tetramethyl-1,3,2-dioxaborolan-2-yl)-1H-pyrazole (1.940 g, 9.321 mmol) in presence of potassium carbonate (3.210 g, 23.301 mmol) and [1,1′-bis(diphenylphosphino)ferrocene]dichloropalladium(II) complex with dichloromethane (0.630 g, 0.776 mmol) in 1,4 dioxane:water (4:1) (38 mL) for 16 h at 100° C. as described in step-4 of example-3. The crude product obtained was purified by combiflash column chromatography (hexanes/ethyl acetate=5/95) to afford the title compound (2.200 g, 73%) as a solid.

### Step-2: (5-(((2-(1-methyl-H-pyrazol-4-yl)pyridin-3-yl)oxy)methyl)-4,5-dihydroisoxazol-3-yl)methanamine hydrochloride (±)

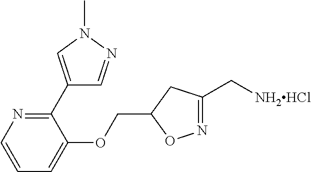

Tert-butyl((5-(((2-(1-methyl-1H-pyrazol-4-yl)pyridin-3-yl)oxy)methyl)-4,5-dihydroisoxazol-3-yl)methyl)carbamate (±) (product of step-1, 2.200 g, 6.495 mmol) was reacted with dioxane.HCl as described in step-5 of example-3 to afford the title compound (1.80 g, 85%).

### Step-3: 1-((5-(((2-(1-methyl-1H-pyrazol-4-yl)pyridin-3-yl)oxy)methyl)-4,5-dihydroisoxazol-3-yl)methyl)-3-(2-methylpyridin-4-yl)urea (±)

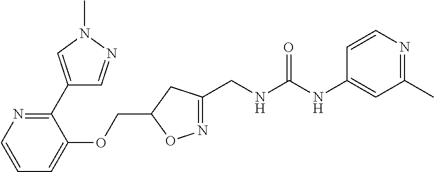 [SMILES: Cc1cc(NC(=O)NCC2=NOC(COc3cccnc3-c3cnn(C)c3)C2)ccn1]

(5-(((2-(1-Methyl-H-pyrazol-4-yl)pyridin-3-yl)oxy)methyl)-4,5-dihydroisoxazol-3-yl)methanamine hydrochloride (±) (product of step-2, 0.200 g, 0.617 mmol) was treated with phenyl(2-methylpyridin-4-yl)carbamate (product of step-1 of example-16, 0.169 g, 0.741 mmol) in presence of triethylamine (0.256 mL, 1.851 mmol) in DMSO (5 mL) as described in step-4 of example-16. The crude product obtained was purified by combiflash column chromatography (dichloromethane/methanol=8/92) to afford the title compound (0.068 g, 26%) as a solid. LCMS: m/z 421.8 [M+H]⁺; ¹HNMR (400 MHz, DMSO-d₆) δ 9.11 (s, 1H), 8.20-8.07 (m, 3H), 8.03 (s, 1H), 7.48-7.41 (dd, J=8.5, 1.4 Hz, 1H), 7.25-7.20 (d, J=2.1 Hz, 1H), 7.19-7.13 (m, 2H), 6.80-6.72 (t, J=5.7, 5.7 Hz, 1H), 5.08-4.98 (td, J=11.2, 11.0, 4.8 Hz, 1H), 4.25-4.11 (qd, J=10.3, 10.1, 10.1, 4.6 Hz, 2H), 4.10-4.02 (d, J=5.4 Hz, 2H), 3.90 (s, 3H), 3.27-3.17 (dd, J=17.5, 10.9 Hz, 1H), 2.96-2.85 (dd, J=17.6, 7.5 Hz, 1H), 2.37 (s, 3H).

### Biology:

### Cytotoxicity Assay in MiaPaCa-2:

MiaPaCa-2 Cells (ATCC) were seeded in 96 well plates (Costar clear flat bottom) at a density of 3000 cells/well and allowed to settle overnight. Test compounds were dissolved in dimethyl sulphoxide (DMSO-Sigma Aldrich, D2650) and incubated with MiaPaCa-2 cells for 72 h. Post 72 h, 50 μl XTT (1 mg/mL; Invitrogen, X 6493) and PMS (8 M; Sigma Aldrich, P9625) reagents were added to the cells in 100 μl of cDMEM (Sigma Aldrich-D5648)+10% FBS (Hyclone-5430088-03)+1×Pen Strep (Sigma Aldrich-P0781). After 1 hour incubation with XTT and PMS absorbance was monitored with Spectramax M3 (Molecular Device). Percentage inhibition of proliferation at 10 μM and 1 μM concentrations was calculated by considering DMSO control as 0% inhibition. IC₅₀ values were plotted using Graph pad prism software (Graph pad Inc, La Jola, Calif., USA).

### NAMPT Fluorescence Assay:

The enzymatic assay was standardized using in-house recombinant NAMPT wild type protein from E. coli expression & NAM (Cat #47865-U) as a substrate. The product NMN formed after enzymatic reaction was derivatized to a fluorescent derivative through sequentially reacting with acetophenone/KOH and formic acid. The derivatized fluorescent NMN derivative was detected at 382 nm excitation wavelength and a 445 nm emission wavelength. The final assay conditions were 50 ng NAMPT, 2 μM Nam (Km conc.), 0.4 mM PRPP (Cat # P8296), 2 mM ATP (Cat # A7699), 0.02% BSA, 2 mM DTT, 12 mM MgCl₂, 50 mM Tris HCl pH-7.5, 2% DMSO (25 L reaction volume: 15 minutes pre-incubation of enzyme with compound & 15 minutes incubation for the complete reaction) in 96 well Black plate. The fluorescence was measured (Excitation at 382 nm & Emission at 445 nm) using Victor³ V fluorimeter. The data is calculated using the RFU values. The final concentration of DMSO was 2% in the assay. Each individual IC₅₀ was determined using 10 point dose response curve generated by GraphPad Prism software Version 4 (San Diego, Calif., USA) using nonlinear regression curve fit for sigmoidal dose response (variable slope).

The compounds were screened at 1 M/10 M concentrations followed by IC₅₀ measurement and the results are summarized in table below along with cell cytotoxicity assay data. The cell cytotoxicity assay data is given in range wherein “+++” refers to an IC₅₀ value less than 50 μM, “++” refers to IC₅₀ value in range of 50.01 μM to 250 μM and “+” refers to IC₅₀ value of greater than 250 μM.

The in-vitro fluorescence assay IC₅₀ (nM) data is also summarized in the table below wherein “A” refers to an IC₅₀ value less than 10 nM, “B” refers to IC₅₀ value in range of 10.01 nM to 25 nM and “C” refers to IC₅₀ value of greater than 25 nM.

| | Cell | Cell | Cell | |
|---|---|---|---|---|
| | cytotoxicity | cytotoxicity | cytotoxicity | Fluorescence |
| | assay | assay | assay | assay |
| Ex. | (% Inhibition | (% Inhibition | IC₅₀ in range | IC₅₀ in range |
| No. | at 1 μM) | at 10 μM) | (μM) | (nM) |
| 1 | — | — | + | A |
| 2 | 95 | 97 | ++ | A |
| 3 | 5 | 80 | + | — |
| 4 | 96 | 96 | +++ | A |
| 4a | 8 | 83 | + | A |
| 4b | 95 | 94 | +++ | A |
| 5 | 47 | 95 | ++ | A |
| 6 | 15 | 80 | + | C |
| 7 | 90 | 90 | ++ | B |
| 7a | — | — | +++ | A |
| 7b | — | — | + | — |
| 8 | — | — | +++ | B |
| 8a | 10 | 10 | − | — |
| 8b | 92 | 94 | ++ | C |
| 9 | 88 | 89 | +++ | B |
| 9a | 12 | 90 | + | A |
| 9b | 95 | 95 | +++ | B |
| 10 | 95 | 95 | ++ | B |
| 10a | 100 | 100 | +++ | — |
| 10b | 100 | 100 | +++ | — |
| 11 | 96 | 95 | +++ | B |
| 11a | 88 | 100 | + | B |
| 11b | 100 | 98 | +++ | B |
| 12 | 96 | 95 | +++ | C |
| 12a | 45 | 96 | + | B |
| 12b | 100 | 100 | +++ | B |
| 13 | 95 | 95 | ++ | A |
| 13a | 20 | 93 | + | C |
| 13b | 100 | 100 | +++ | B |
| 14 | 95 | 95 | +++ | B |
| 14a | 93 | 98 | + | — |
| 14b | 100 | 98 | +++ | — |
| 15 | 99 | 99 | ++ | — |
| 16 | 89 | 89 | ++ | A |
| 17 | 90 | 89 | +++ | A |
| 18 | 90 | 89 | ++ | C |
| 19 | 17 | 82 | + | B |

1. A compound of formula (I): 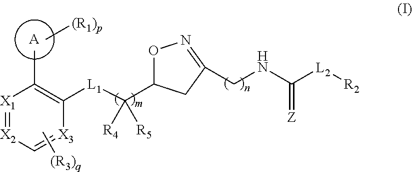
or a pharmaceutically acceptable salt or a stereoisomer thereof;
wherein,
ring A is carbocyclyl or heterocyclyl;
X₁, X₂ and X₃ independently are C or N; provided all X₁, X₂ and X₃ are not C at any instance;
L₁ is —O—, —S—, —SO—, —SO₂—, —NH—, —CO—, —NHCO—, —CONH—, —NHSO₂— or —SO₂NH—;
L₂ is a direct bond, —CH═CH— or —NR_{b}(CH₂)ᵣ—;
Z is O, S or NCN;
R₁ at each occurrence is independently alkyl, nitro, halo, haloalkyl, hydroxyalkyl, —ORₐ, —(CH₂)ₙNR_{b}R_{c}, —(SO₂)-alkyl, —(SO₂)—NR_{b}R_{c}, —NH(CO)alkyl or —(CO)NHR_{b};
R₂ is optionally substituted carbocyclyl or optionally substituted heterocyclyl wherein the optional substituent is R;
R₃ at each occurrence is hydrogen, halo, amino, nitro, cyano, alkyl, hydroxy, alkoxy, haloalkyl or haloalkoxy;
R₄ and R₅ independently are hydrogen or alkyl;
alternatively, R₄ and R₅ together with the carbon atom to which they are attached formr an optionally substituted cycloalkyl ring; wherein the optional substituent is alkyl or halo;
R₆ at each occurrence is independently one or more alkyl, amino, halo, nitro, cyano, haloalkyl, hydroxyl, alkoxy, —NHSO₂-alkyl, cycloalkyl, optionally substituted aryl or optionally substituted heteroaryl wherein the optional substituent is halo, alkyl, hydroxyl, alkoxy or haloalkyl;
Rₐ is hydrogen, alkyl or arylalkyl;
R_{b} and R_{c} independently are hydrogen or alkyl;
m, n and p independently are 0, 1, 2 or 3;
q is 1, 2 or 3; and
r is 0, 1 or 2.

2. The compound according to claim 1 is a compound of formula (IA): 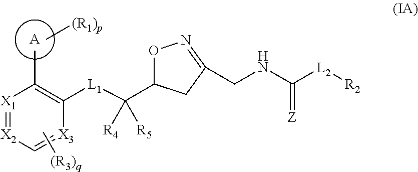
or a pharmaceutically acceptable salt or a stereoisomer thereof;
wherein,
ring A, L₁, L₂, X₁, X₂, X₃, Z, R₁, R₂, R₃, R₄, R₅, p and q are same as defined in claim 1.

3. The compound according to claim 1 is a compound of formula (IB): 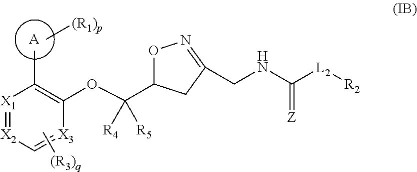
or a pharmaceutically acceptable salt or a stereoisomer thereof;
wherein,
R₄ and R₅ are hydrogen, ring A, L₂, X₁, X₂, X₃, Z, R₁, R₂, R₃, p and q are same as defined in claim 1.

4. The compound according to claim 1 is a compound of formula (IC): 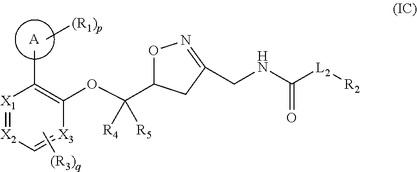
or a pharmaceutically acceptable salt or a stereoisomer thereof;
wherein,
R₄ and R₅ are hydrogen, ring A, L₂, X₁, X₂, X₃, R₁, R₂, R₃, p and q are same as defined in claim 1.

5. The compound according to claim 1 is a compound of formula (ID): 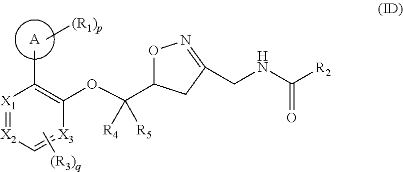
or a pharmaceutically acceptable salt or a stereoisomer thereof;
wherein,
R₄ and R₅ are hydrogen, ring A, X₁, X₂, X₃, R₁, R₂, R₃, p and q are same as defined in claim 1.

6. The compound according to claim 1 is a compound of formula (IE): 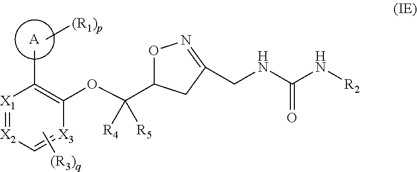
or a pharmaceutically acceptable salt or a stereoisomer thereof;
wherein,
R₄ and R₅ are hydrogen, ring A, X₁, X₂, X₃, R₁, R₂, R₃, p and q are same as defined in claim 1.

7. The compound according to claim 1 is a compound of formula (IF): 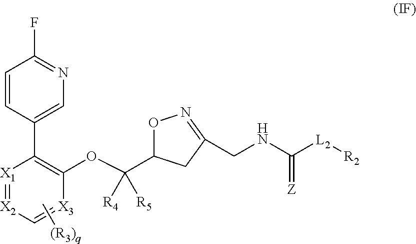
or a pharmaceutically acceptable salt or a stereoisomer thereof;
wherein,
L₂, X₁, X₂, X₃, Z, R₂, R₃, R₄, R₅, and q are same as defined in claim 1.

8. The compound according to claim 1 is a compound of formula (IG): 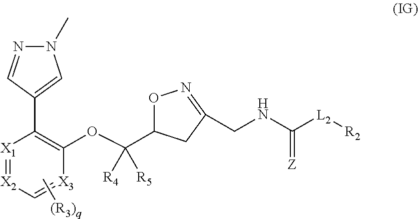
or a pharmaceutically acceptable salt or a stereoisomer thereof;
wherein,
L₂, X₁, X₂, X₃, Z, R₂, R₃, R₄, R₅ and q are same as defined in claim 1.

9. The compound according to claim 1,
wherein ring A is phenyl, pyridinyl or pyrazolyl.

10. The compound according to claim 1, wherein R₂ is optionally substituted pyridinyl, optionally substituted pyridazinyl or optionally substituted imidazo[1,2-a]pyridinyl.

11. The compound according to claim 1 is a compound of formula (IH): 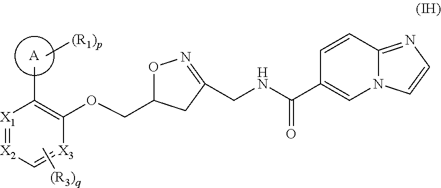
or a pharmaceutically acceptable salt or a stereoisomer thereof;
wherein,
ring A, X₁, X₂, X₃, R₁, R₃, p and q are same as defined in claim 1.

12. The compound according to claim 1, wherein the ring 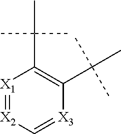
is

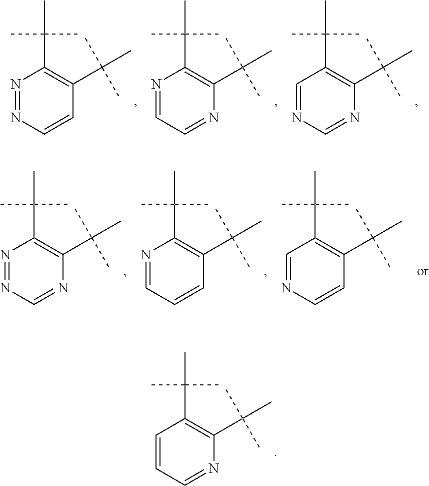

13-15. (canceled)

16. A compound according to claim 1 selected from the group consisting of:

| Ex | |
|---|---|
| No. | IUPAC NAME |
| 1 | N-((5-(((3-(1-methyl-1H-pyrazol-4-yl)pyridin-2-yl)oxy)methyl)-4,5- |
| | dihydroisoxazol-3-yl)methyl)imidazo[1,2-a]pyridine-6-carboxamide (±); |
| 2 | N-((5-(((6′-fluoro-[3,3′-bipyridin]-2-yl)oxy)methyl)-4,5-dihydroisoxazol-3- |
| | yl)methyl)-imidazo[1,2-a]pyridine-6-carboxamide (±); |
| 3 | N-((5-(((6′-fluoro-[3,3′-bipyridin]-4-yl)oxy)methyl)-4,5-dihydroisoxazol-3- |
| | yl)methyl)-imidazo[1,2-a]pyridine-6-carboxamide (±); |
| 4 | N-((5-(((6′-fluoro-[2,3′-bipyridin]-3-yl)oxy)methyl)-4,5-dihydroisoxazol-3- |
| | yl)methyl)imidazo[1,2-a]pyridine-6-carboxamide (±); |
| 4a | N-((5-(((6′-fluoro-[2,3′-bipyridin]-3-yl)oxy)methyl)-4,5-dihydroisoxazol-3- |
| | yl)methyl)imidazo[1,2-a]pyridine-6-carboxamide ( [symbol] somer-1); |
| 4b | N-((5-(((6′-fluoro-[2,3′-bipyridin]-3-y])oxy)methyl)-4,5-dihydroisoxazol-3- |
| | yl)methyl)imidazo[1,2-a]pyridine-6-carboxamide (Isomer-2); |
| 5 | N-((5-(((2-(1-methyl-1H-pyrazol-4-yl)pyridin-3-yl)oxy)methyl)-4,5- |
| | dihydroisoxazol-3-yl)methyl)imidazo[1,2-a]pyridine-6-carboxamide (±); |
| 6 | N-((5-(((2-(3-fluoro-4-(methylcarbamoyl)phenyl)pyridin-3-yl)oxy)methyl)-4,5- |
| | dihydroisoxazol-3-yl)methyl)imidazo[1,2-a]pyridine-6-carboxamide (±); |
| 7 | N-((5-(([2,3′-bipyridin]-3-yloxy)methyl)-4,5-dihydroisoxazol-3-yl)methyl)- |
| | imidazo[1,2-a]pyridine-6-carboxamide (±); |
| 7a | N-((5-(([2,3′-bipyridin]-3-yloxy)methyl)-4,5-dihydroisoxazol-3-yl)methyl)- |
| | imidazo[1,2-a]pyridine-6-carboxamide (Isomer-1); |
| 7b | N-((5-(([2,3′-bipyridin]-3-yloxy)methyl)-4,5-dihydroisoxazol-3-yl)methyl)- |
| | imidazo[1,2-a]pyridine-6-carboxamide (Isomer-2); |
| 8 | N-((5-(((6′-chloro-[2,3′-bipyridin]-3-yl)oxy)methyl)-4,5-dihydroisoxazol-3- |
| | yl)methyl)imidazo[1,2-a]pyridine-6-carboxamide(±); |
| 8a | N-((5-(((6′-chloro-[2,3′-bipyridin]-3-yl)oxy)methyl)-4,5-dihydroisoxazol-3- |
| | yl)methyl)imidazo[1,2-a]pyridine-6-carboxamide (Isomer-1); |
| 8b | N-((5-(((6′-chloro-[2,3′-bipyridin]-3-yl)oxy)methyl)-4,5-dihydroisoxazol-3- |
| | yl)methyl)imidazo[1,2-a]pyridine-6-carboxamide (Isomer-2); |
| 9 | N-((5-(((5,6′-difluoro-[2,3′-bipyridin]-3-yl)oxy)methyl)-4,5-dihydroisoxazol-3- |
| | yl)methyl)imidazo[1,2-a]pyridine-6-carboxamide(±); |
| 9a | N-((5-(((5,6′-difluoro-[2,3′-bipyridin]-3-yl)oxy)methyl)-4,5-dihydroisoxazol-3- |
| | yl)methyl)imidazo[1,2-a]pyridine-6-carboxamide (Isomer-1); |
| 9b | N-((5-(((5,6′-difluoro-[2,3′-bipyridin]-3-yl)oxy)methyl)-4,5-dihydroisoxazol-3- |
| | yl)methyl)imidazo[1,2-a]pyridine-6-carboxamide (Isomer-2); |
| 10 | N-((5-(((6-cyano-6′-fluoro-[2,3′-bipyridin]-3-yl)oxy)methyl)-4,5-dihydroisoxazol- |
| | 3-yl)methyl)imidazo[1,2-a]pyridine-6-carboxamide(±); |
| 10a | N-((5-(((6-cyano-6′-fluoro-[2,3′-bipyridin]-3-yl)oxy)methyl)-4,5-dihydroisoxazol- |
| | 3-yl)methyl)imidazo[1,2-a]pyridine-6-carboxamide (Isomer-1); |
| 10b | N-((5-(((6-cyano-6′-fluoro-[2,3′-bipyridin]-3-yl)oxy)methyl)-4,5-dihydroisoxazol- |
| | 3-yl)methyl)imidazo[1,2-a]pyridine-6-carboxamide (Isomer-2); |
| 11 | N-((5-(((6′-fluoro-5-methyl-[2,3′-bipyridin]-3-yl)oxy)methyl)-4,5-dihydroisoxazol-3- |
| | yl)methyl)imidazo[1,2-a]pyridine-6-carboxamide(±); |
| 11a | N-((5-(((6′-fluoro-5-methyl-[2,3′-bipyridin]-3-yl)oxy)methyl)-4,5-dihydroisoxazol-3- |
| | yl)methyl)imidazo[1,2-a]pyridine-6-carboxamide (Isomer-1); |
| 11b | N-((5-(((6′-fluoro-5-methyl-[2,3′-bipyridin]-3-yl)oxy)methyl)-4,5-dihydroisoxazol-3- |
| | yl)methyl)imidazo[1,2-a]pyridine-6-carboxamide (Isomer-2); |
| 12 | N-((5-(((6′-fluoro-6-methyl-[2,3′-bipyridin]-3-yl)oxy)methyl)-4,5-dihydroisoxazol- |
| | 3-yl)methyl)imidazo[1,2-a]pyridine-6-carboxamide(±); |
| 12a | N-((5-(((6′-fluoro-6-methyl-[2,3′-bipyridin]-3-yl)oxy)methyl)-4,5-dihydroisoxazol- |
| | 3-yl)methyl)imidazo[1,2-a]pyridine-6-carboxamide (Isomer-1); |
| 12b | N-((5-(((6′-fluoro-6-methyl-[2,3′-bipyridin]-3-yl)oxy)methyl)-4,5-dihydroisoxazol- |
| | 3-yl)methyl)imidazo[1,2-a]pyridine-6-carboxamide (Isomer-2); |
| 13 | N-((5-(((5-chloro-[2,3′-bipyridin]-3-yl)oxy)methyl)-4,5-dihydroisoxazol-3- |
| | yl)methyl)imidazo[1,2-a]pyridine-6-carboxamide (±); |
| 13a | N-((5-(((5-chloro-[2,3′-bipyridin]-3-yl)oxy)methyl)-4,5-dihydroisoxazol-3- |
| | yl)methyl)imidazo[1,2-a]pyridine-6-carboxamide (Isomer-1); |
| 13b | N-((5-(((5-chloro-[2,3′-bipyridin]-3-yl)oxy)methyl)-4,5-dihydroisoxazol-3- |
| | yl)methyl)imidazo[1,2-a]pyridine-6-carboxamide (Isomer-2); |
| 14 | N-((5-(((5-chloro-6′-fluoro-[2,3′-bipyridin]-3-yl)oxy)methyl)-4,5-dihydroisoxazol- |
| | 3-yl)methyl)imidazo[1,2-a]pyridine-6-carboxamide (±); |
| 14a | N-((5-(((5-chloro-6′-fluoro-[2,3′-bipyridin]-3-yl)oxy)methyl)-4,5-dihydroisoxazol- |
| | 3-yl)methyl)imidazo[1,2-a]pyridine-6-carboxamide (Isomer-1); |
| 14b | N-((5-(((5-chloro-6′-fluoro-(2,3′-bipyridin]-3-yl)oxy)methyl)-4,5-dihydroisoxazol- |
| | 3-yl)methyl)imidazo[1,2-a]pyridine-6-carboxamide (Isomer-2); |
| 15 | N-((5-(((5-fluoro-[2,3′-bipyridin]-3-yl)oxy)methyl)-4,5-dihydroisoxazol-3- |
| | yl)methyl)imidazo[1,2-a]pyridine-6-carboxamide (±); |
| 16 | 1-((5-(((6′-fluoro-[2,3′-bipyridin]-3-yl)oxy)methyl)-4,5-dihydroisoxazol-3- |
| | yl)methyl)-3-(2-methylpyridin-4-yl)urea (±); |
| 17 | 1-((5-(((6′-fluoro-[2,3′-bipyridin]-3-yl)oxy)methyl)-4,5-dihydroisoxazol-3- |
| | yl)methyl)-3-(pyridin-4-yl)urea (±); |
| 18 | 1-((5-(((6′-fluoro-[2,3′-bipyridin]-3-yl)oxy)methyl)-4,5-dihydroisoxazol-3- |
| | yl)methyl)-3-(pyridazin-4-yl)urea(±); and |
| 19 | 1-((5-(((2-(1-methyl-1H-pyrazol-4-yl)pyridin-3-yl)oxy)methyl)-4,5- |
| | dihydroisoxazol-3-yl)methyl)-3-(2-methylpyridin-4-yl)urea(±). |

[symbol]

indicates data missing or illegible when filed
or a pharmaceutically acceptable salt or a stereoisomer thereof.

17. A pharmaceutical composition, comprising at least one compound according to claim 1, or a pharmaceutically acceptable salt or a stereoisomer thereof, and a pharmaceutically acceptable carrier or excipient.

18. The pharmaceutical composition according to claim 17, further comprising at least one additional pharmaceutical agent wherein the said additional pharmaceutical agent is an anticancer agent, a chemotherapy agent, an immunosuppressant agent, a pain relieving agent or an anti-proliferative agent.

19-21. (canceled)

22. A method of inhibiting nicotinamide phosphoribosyl transferase (NAMPT) in a subject, comprising administering to the subject a therapeutically effective amount of a compound according to claim 1.

23. (canceled)

24. A method of treating a NAMPT mediated disorder or disease in a subject, comprising administering to the subject a therapeutically effective amount of a compound according to claim 1.

25. The method according to claim 17, wherein the NAMPT mediated disorder is an immune disorder or an inflammatory disorder.

26. The method according to claim 17, wherein the NAMPT mediated disorder is cancer.

27. The method according to claim 17, wherein the said disorder or disease include, but not limited to the group consisting of cancer, pancreatic cancer, ovarian cancer, lung cancer, prostate cancer, skin cancer, breast cancer, uterine cancer, renal cancer, head and neck cancer, brain cancer, colon cancer, cervical cancer, bladder cancer, leukemia, lymphoma, Hodgkin's disease, viral infections including adult respiratory distress syndrome, ataxia telengiectasia, Human Immunodeficiency Virus, hepatitis virus, herpes virus, herpes simplex, inflammatory disorders, irritable bowel syndrome, inflammatory bowel disease, rheumatoid arthritis, asthma, chronic obstructive pulmonary disease, osteoarthritis, osteoporosis, fibrotic diseases, dermatosis, atopic dermatitis, psoriasis, ultra-violet induced skin damage, systemic lupus erythematosis, multiple sclerosis, psoriatic arthritis, ankylosing spondylitis, graft-versus-host disease, Alzheimer's disease, cerebrovascular accident, atherosclerosis, restenosis, diabetes, glomerulonephiritis, metabolic syndrome, non-small cell lung cancer, small cell lung cancer, multiple myeloma, leukemias, lymphomas, cancers of the brain and central nervous system, squamous cell cancers, kidney cancer, uretral and bladder cancers, cancers of head and neck.
